(19) United States
(12) Patent Application Publication
(10) Pub. No.: US 2011/0269680 A1
(43) Pub. Date: Nov. 3, 2011

(54) GLP-1 ANALOGS AND USES THEREOF

(75) Inventors: Rajesh Jain, New Delhi (IN); Virender Kumar Vinayak, New Delhi (IN); Sudhanand Prasad, New Delhi (IN)

(73) Assignee: PANACEA BIOTEC LIMITED, New Delhi (IN)

(21) Appl. No.: 13/142,516

(22) PCT Filed: Dec. 24, 2009
(86) PCT No.: PCT/IN2009/000742

§ 371 Date: Jun. 28, 2011

(30) Foreign Application Priority Data
Dec. 29, 2008 (IN) 2956/DEL/2008

(51) Int. Cl. A61K 38/26; A61P 3/10; C07K 14/605

(52) U.S. Cl. 514/7.2; 530/308; 514/11.7

(57) ABSTRACT

The present invention relates to a peptide selected from a group comprising GLP-1 analogs, its truncated forms, or pharmaceutically acceptable salts and derivatives thereof. The present invention further relates to a peptide selected from a group comprising GLP-1 analogs, its truncated forms, or pharmaceutically acceptable salts and derivatives thereof, wherein one or more aminoacids are substituted with a non-coded aminoacid.

### FIELD OF INVENTION

The present invention relates to a peptide selected from a group comprising GLP-1 analogs, its truncated forms, or pharmaceutically acceptable salts and derivatives thereof. The present invention further relates to a peptide selected from a group comprising GLP-1 analogs, its truncated forms, or pharmaceutically acceptable salts and derivatives thereof, wherein one or more aminoacids are substituted with a non-coded aminoacid.

### BACKGROUND OF INVENTION

Type-II diabetes is increasingly becoming a worldwide epidemic. Inadequate secretion of insulin may be a very early element in the development of Type-II diabetes and that its progression is due to declining β-cell function [Pratley R E, Weyer C: Diabetologia 44:929-945, 2001. Weyer C et al; J Clin Invest 104:787-794, 1999. Kahn S E: Am J Med 108 (Suppl. 6a):2S-8S, 2000]. The β-cell defect is partly due to loss of β-cells, but the loss which may amount to 50% in advanced type-II diabetes [Butler A E et al; Diabetes 52:102-110, 2003], does not seem to parallel the dysfunction. Insulin secretion after the ingestion of a mixed meal is stimulated not only by the rise in glucose concentrations but also by the secretion of incretin hormones, namely glucagon-like peptide-1 (GLP-1) and gastric inhibitory polypeptide (GIP; also referred to as glucose-dependent insulinotropic polypeptide), from the gut [Creutzfeldt W, Nauck M: Diabetes Metab Rev 8:149-177, 1992]. Both hormones are currently considered for the treatment of type-II diabetes because of their glucose lowering activity [Creutzfeldt W, Nauck M: Diabetes Metab Rev 8:149-177, 1992; Meier J J et al; BioDrugs 17:93-102, 2003].

Glucagon-Like peptide-1 (GLP-1) was discovered in 1984. GLP-1 is a neuroendocrine hormone of the distal gut with a strong insulinotropic action that is synthesized and secreted from L-cells in the intestine in response to meal ingestion [Kieffer T J, Habener J F 1999. Endocr Rev 20: 876-913]. Importantly, the action of GLP-1 is glucose-dependent, avoiding the occurrence of hypoglycemia. The intracellular precursor to GLP-1, GLP-1-(1-37), is cleaved from proglucagon, and the first six aminoacids are subsequently removed from the N terminus to form bioactive peptides. About 80% of truncated GLP-1 is amidated to form GLP-1(7-36) amide; the predominant secreted form of GLP-1, whereas the remainder is released as GLP-1-(7-37) [Orskov C. et al; Diabetes 43:535-539, 1994].

Both GLP-1(7-36) NH₂ and GLP-1(7-37) interact with a specific GLP-1 receptor (GLP-1r) that is expressed on the pancreatic β-cell, and in other tissues such as the gastrointestinal tract and central nervous system. In vivo, GLP-1(7-36) NH₂ and GLP-1(7-37) have equipotent effects to stimulate glucose-stimulated insulin release, [Orskov C. et al; Diabetes 43:535-539, 1994], and a physiological role for these hormones in the incretin response has been established in several animal models. Numerous effects other than stimulation of insulin release have been ascribed to GLP-1. In pancreas, it stimulates insulin biosynthesis, restoration of glucose sensitivity to the islets and stimulates increased expression of the glucose transporter GLUT-2 and glucokinase. GLP-1 regulates β cell mass by stimulating replication and growth and also inhibits apoptosis of existing β cells and neogenesis of new β-cells from duct precursor cells. GLP-1 inhibits glucagon secretion and leads to reduced hepatic glucose output. In the gut GLP-1 is a potent inhibitor of motility and gastric emptying and also inhibits gastric acid secretion. This leads to decreased food intake and reduced body weight [Stoffers, D. A. et al; Diabetes 2000, 49, 741-748. Drucker D J Diabetes Care 26:2929-2940, (2003)].

GLP-1 acts through a G protein-coupled receptor to exert its functions. This receptor is expressed in many tissues, including pancreatic islets, the central nervous system, lung, kidney, heart, and the gut. GLP-1 is coupled to its receptor through stimulatory Gα and adenylyl cyclase to increase intracellular cAMP. GLP-1 can induce other intracellular signals as well, including increases in intracellular calcium, phosphoinositol 3-kinase (PI3K) activity, and mitogen-activated protein kinase activity [Buteau J. et al; Diabetologia 42:856-864, 1999; Bullock B P. et al; Endocrinology 137: 2968-2978, 1996].

However, the drawback to the use of GLP-1 is its short half-life of about 2 minutes because of very rapid enzymatic degradation of GLP-1 by dipeptidyl peptidase-IV (DPP-IV) and Neutral Endopeptidase 24.11 (NEP 24.11).

DPP-IV is a ubiquitous cell surface and circulating enzyme found in large amounts at the brush border of kidney epithelium [Deacon C F et al; J Clin Endocrinol Metab 80:952-957, 1995]. Degradation of GLP-1 by DPP-IV seems to require alanine or proline at the second N-terminal position. There have been several reports of GLP-1 homologs with N-terminal modifications ranging from substitution of the alanine with threonine, serine, valine, aminoisobutyric acid or glycine as well as glycation of the alanine. To protect against cleavage by DPP-IV biological activity of a novel GLP-1 analog in which 6-aminohexanoic acid (Aha) is inserted between the histidine and alanine at positions 7 and 8.

NEP 24.11 is a membrane-bound zinc metallopeptidase that cleaves peptides at the NH2-terminal side of aromatic or hydrophobic aminoacids, and six potential cleavage sites in GLP-1 were identified. GIP was also degraded by NEP 24.11, albeit more slowly, and it was suggested that its larger size, 42 vs. 30 aminoacids for GLP-1, may be one factor determining its suitability as a substrate, since the enzyme has a preference for smaller peptides, but the physiological significance was not examined in vivo. Since NEP 24.11 has a widespread tissue distribution and is found in particularly high concentration in the kidneys, it could be speculated to be involved in the renal clearance of peptide hormones.

Two main intervention strategies are under development to prevent degradation of GLP-1 i.e. specific inhibitors of DPP-IV and subtle modifications of the GLP-1 molecule to generate analogs that are resistant to DPP IV. Although structural modification of GLP-1 may overcome degradation by DPP IV, this does not address the loss of GLP-1 by renal filtration [Meier J J. et al; Diabetes 53:654-662, 2004]. People have attempted to prevent renal filtration of GLP-1 by acylation (attaching long-chain fatty acid molecules) [Meier J J. et al; Diabetes 53:654-662, 2004]. Acylating peptides facilitate binding to plasma proteins, such as albumin, thereby minimizing their elimination by the kidney. LY315902 (Eli Lilly & Co., Indianapolis, Ind.), for example, is an acylated GLP-1 analog with an octanoyl fatty acid chain. NN2211 (Liraglutide; Novo Nordisk, Bagsvaerd, Denmark) contains a hexadecanoyl fatty acid group attached to the ε-amino group of Lys26, and CJC-1131 (Conjuchem) contains a reactive chemical linker attached to the amino group of Lys34 [Green B D et al; Biol. Chem 385:169-177, 2004]. NN2211 and CJC-1131 show sustained activities and increased half lives in excess of 8 h. Other attempts to acylate GLP-1 with palmitate (18-carbon fatty acid) produce analogs with moderately prolonged activities but with greatly reduced bioavailability.

Dialkylated aminoacids (Daa or α,α-dialkylated aminoacids) are known to induce conformational constrain in the peptide backbone. The conformational characteristics of α,α-dialkylated aminoacids have been well studied. The incorporation of these aminoacids restricts the rotation of φ, ψ angles within the molecule, thereby stabilizing a desired peptide conformation. The prototypic member of α,α-dialkylated aminoacids, α-aminoisobutyric acid (Aib) or α,α-dimethyl glycine has been shown to induce β-turn or helical conformation when incorporated in a peptide sequence (Prasad B. V. V. and Balaram, P. 1984, CRC Crit. Rev. Biochem. 16, 307-347; Karle, I. L. and Balaram, P. 1990 Biochemistry, 29, 6747-6756). As per the prior arts cited above native GLP-1 is degraded in vivo by dipeptidyl peptidase IV (DPP-IV), which releases the N-terminal dipeptide, His-Ala, and inactivates GLP-1, and the half-life of GLP-1 in blood is only few minutes. Therefore its clinical application was so difficult.

WO2007/124461 discloses a variety of GLP-1 compounds having better half life that comprise a GLP-1 analog, having one or more of the following characteristics: 1) amino acid substitutions at particular locations of GLP-1, 2) added amino acids at the N-terminus and/or the C-terminus of GLP-1, 3) absence of amino acids at the N-terminus and/or the C-terminus of GLP-1, and/or 4) presence of a ring formed by joining the side chains of specific amino acids with the polypeptide. The application does not disclose or suggest the substitution of natural aminoacid of parent GLP-1 with sugar aminoacid or furan aminoacid.

EP1961764A relates to GLP-1 derivative with longer half-life, wherein the glycosylated aminoacid is used in the native GLP-1 molecule for inducing resistance to degradative enzyme like DPP-IV. The patent application does not disclose or suggest the substitution of natural aminoacid of parent GLP-1 with sugar aminoacid, dialkylated amino or furan aminoacid at specific positions.

WO2006/010143A relates to the method of prolongation of half life of GLP-1 compounds by enzymatic glyco-conjugation reactions. The patent application does not disclose or suggest the substitution of natural aminoacid of parent GLP-1 with sugar aminoacid, dialkylated aminoacid or furan amino acid at specific positions.

WO2006/037810A2 relates to modified GLP-1 analogues acylated with a diacid. The patent application does not disclose or suggest the substitution of natural aminoacid of parent GLP-1 with sugar aminoacid, dialkylated aminoacid or furan aminoacid at specific positions.

US 20080096819 relates to methods, compositions and kits relating to modified molecules comprising one or more amino acid substitutions or additions with a naturally occurring amino acid, one or more amino acid substitutions with a non-naturally occurring amino acid, and a chemical moiety added to said non-natural amino acid residue. The patent application does not disclose or suggest the substitution of natural aminoacid of parent GLP-1 with sugar aminoacid, dialkylated aminoacid or furan aminoacid at specific positions.

In the past, many GLP-1 derivatives have been reported, which acquired resistance to DPP-IV by substitution and/or modification of aminoacid residues around the cleaved site by DPP-IV, but still there exists a need of more stabilized GLP-1 and related peptides with longer half life in plasma and better resistance to DPP-IV and NEP enzyme.

Inventors of the present invention have surprisingly found that the substitution of natural aminoacids of parent GLP-1 and related peptide with non-coded aminoacids like sugar aminoacids, furan aminoacids and dialkylated aminoacids in a site specific manner, improves enzymatic stability against DPP-IV and NEP. Inventors of the present invention have also found that the dialkylated aminoacids provides structural robustness to the GLP-1 peptides which results in better stabilization.

### SUMMARY OF THE INVENTION

The present invention relates to a peptide selected from a group comprising GLP-1 analogs, its truncated forms, or pharmaceutically acceptable salts and derivatives thereof.

The present invention further relates to a peptide selected from a group comprising GLP-1 analogs, its truncated forms, or pharmaceutically acceptable salts and derivatives thereof, wherein one or more aminoacids are substituted with a non-coded aminoacid.

The present invention further relates to a peptide selected from a group comprising GLP-1 analogs, its truncated forms, or pharmaceutically acceptable salts and derivatives thereof, wherein one or more aminoacids are substituted with non-coded aminoacids selected from the group comprising sugar aminoacids, furan aminoacids and dialkylated aminoacids.

The present invention further relates to a peptide having the general formula:

(Seq ID No. 13)
His-Xaa¹-Xaa²-Gly-Thr-Phe-Thr-Ser-Xaa³-Xaa⁴-Xaa⁵⁻
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention also relates to a peptide having the general formula:

X′aa¹-X′aa²-X′aa³-X′aa⁴-X′aa⁵-X′aa⁶-His-Xaa¹-Xaa²-
Gly-Thr-Phe-Thr-Ser-Xaa3-Xaa⁴-Xaa⁵⁻Ser-Tyr-Xaa⁶-
Glu-Gly-Gln-Ala-Ala-Lys-Xaa⁷-Phe-Ile-Xaa⁸-Trp-
Leu-Val-Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

### BRIEF DESCRIPTION OF THE DRAWINGS

FIG. 1) provides blood glucose levels produced after administration of GLP-1(7-37) parent, saline solution (control negative) and peptides of Seq ID No.03, Seq ID No.05, and Seq ID No.12 using the Oral Glucose Tolerance Test (OGTT), in C57BL/6 normal 6 week old male mice at 2 μg/mouse dose.

FIG. 2) provides blood glucose levels produced after administration of GLP-1(7-36) parent, saline solution (control negative) and peptide of Seq ID No.06 using the Oral Glucose Tolerance Test (OGTT), in C57BL/6 normal 8 week old female mice at 2 μg/mouse dose.

FIG. 3) provides blood glucose levels produced after administration of GLP-1(7-37) parent, saline solution (control negative), and peptides of Seq ID No.01, Seq ID No.02, Seq ID No.04, Seq ID No.07, Seq ID No.08, Seq ID No.09 and Seq ID No.10 using the Oral Glucose Tolerance Test (OGTT), in C57BL/6 normal 6 week old male mice at 2 μg/mouse dose.

FIG. 4) provides percent reduction in blood glucose (AUC₀₋₆₀) after administration of different peptides (Seq ID No.01 to 10 & 11) [OGTT Experiment in C57BL/6 mice] at 2 μg/mouse dose.

FIG. 5) provides blood glucose levels produced after administration of GLP-1(7-37) parent, saline solution (control negative) and peptides of Seq ID No.02, Seq ID No.03, Seq ID No.04, and Seq ID No.11, using the Oral Glucose Tolerance Test (OGTT), in C57BL/6 normal 6 week old male mice at 400 nmol/kg dose.

FIG. 6) provides blood glucose levels produced after administration of GLP-1(7-37) parent, saline solution (control negative) and peptides of Seq ID No.03 and Seq ID No.04, using the Oral Glucose Tolerance Test (OGTT), in C57BL/6 normal 6 week old male mice at 200 nmol/kg dose.

FIG. 7) provides blood glucose levels produced after administration of GLP-1(7-37) parent, saline solution (control negative) and peptide of Seq ID No.03, in male mice using the Oral Glucose Tolerance Test (OGTT), in C57BL/6 normal 6 week old male mice at 100 nmol/kg dose.

FIG. 8) provides blood glucose levels produced after administration of glucose level of GLP-1(7-37) parent, saline solution (control) and peptide of Seq ID No.03 in male mice using the Oral Glucose Tolerance Test (OGTT), in C57BL/6 normal 6 week old male mice at 25 nmol/kg dose.

FIG. 9) provides percent reduction in blood glucose (AUC₀₋₁₂₀) after administration of GLP-1 (7-37) parent and peptides of (Seq. ID No. 02, Seq. ID No. 03, Seq. ID No. 04 and Seq. ID No. 11) at dose (400 nmol, 200 nmol, 100 nmol and 25 nmol/kg)

FIG. 10) provides glucose response curve produced after administration of peptide of Seq ID No. 01 at different doses (100 nmol/kg, 25 nmol/kg, 10 nmol/kg, 5 nmol/kg and 1 nmol/kg) and saline solution (Control negative) using the Oral Glucose Tolerance Test (OGTT), in C57BL/6 normal male mice (6 wk)

FIG. 11) provides the percent reduction in blood glucose (AUC₀₋₆₀) after administration of peptide of Seq ID No. 01 at different doses (400 nmol, 200 nmol, 100 nmol and 25 nmol/kg).

FIG. 12) provides blood glucose levels after administration of peptide of Seq ID No. 01 in 42 days study in 6-8 week old db/db mice (25 nmol/kg)

FIG. 13) provides blood glucose levels after administration of peptide of Seq ID No. 01 in 35 days study in 10-12 week old db/db mice (25 nmol/kg)

### DETAILED DESCRIPTION OF THE INVENTION

The present invention relates to a peptide selected from a group comprising GLP-1 analogs, its truncated forms, or pharmaceutically acceptable salts and derivatives thereof.

The present invention further relates to a peptide selected from a group comprising GLP-1 analogs, its truncated forms or pharmaceutically acceptable salts and derivatives thereof, wherein one or more aminoacids are substituted with a non-coded aminoacid.

The present invention further relates to a peptide selected from a group comprising GLP-1 analogs, its truncated forms, or pharmaceutically acceptable salts and derivatives thereof, wherein one or more aminoacids are substituted with non-coded aminoacids selected from the group comprising sugar aminoacids, furan aminoacids and dialkylated aminoacids.

The present invention relates to a peptide selected from a group comprising of analogs of GLP-1 (1-37), GLP-1(7-34), GLP-1(7-35), GLP-1 (6-37), GLP-1(7-37), GLP-1(7-37) amide, GLP-1(7-36), GLP-1(7-36) amide, their truncated forms, or pharmaceutically acceptable salts and derivatives thereof, wherein one or more aminoacids are substituted with a non-coded aminoacid.

The present invention relates to a peptide selected from a group comprising of analogs of GLP-1 (1-37), GLP-1(7-34), GLP-1(7-35), GLP-1 (6-37), GLP-1(7-37), GLP-1(7-37) amide, GLP-1(7-36), GLP-1(7-36) amide, their truncated forms, or a pharmaceutically acceptable salt and derivatives thereof, wherein one or more aminoacids are substituted with a non-coded aminoacids selected from the group comprising sugar aminoacids, furan aminoacids and dialkylated aminoacids.

The present invention also relates to a pharmaceutical composition comprising a peptide selected from a group comprising GLP-1 analogs, its truncated forms or pharmaceutically acceptable salts and derivatives thereof, wherein one or more aminoacids are substituted with a non-coded aminoacid, and a pharmaceutically acceptable carrier.

The present invention also relates to a pharmaceutical composition comprising a peptide selected from a group comprising GLP-1 analogs, its truncated forms, or pharmaceutically acceptable salts and derivatives thereof, wherein one or more aminoacids are substituted with a non-coded aminoacid selected from the group comprising sugar aminoacids, furan aminoacids and dialkylated aminoacids; and a pharmaceutically acceptable carrier.

The present invention further relates to a process for synthesis of peptide selected from a group comprising GLP-1 analogs, its truncated forms or pharmaceutically acceptable salts and derivatives thereof, wherein one or more aminoacids are substituted with a non-coded aminoacid.

The present invention further relates to a process for synthesis of peptide selected from a group comprising GLP-1 analogs, its truncated forms or pharmaceutically acceptable salts and derivatives thereof, wherein one or more aminoacids are substituted with a non-coded aminoacid selected from the group comprising sugar aminoacids, furan aminoacids and dialkylated aminoacids.

The present invention also relates to a method of eliciting an agonist effect from a GLP-1 receptor comprising administering a peptide selected from a group comprising GLP-1 analogs, its truncated forms or pharmaceutically acceptable salts and derivatives thereof, wherein one or more aminoacids are substituted with a non-coded aminoacid.

The present invention also relates to a method of eliciting an agonist effect from a GLP-1 receptor comprising administering a peptide selected from a group comprising GLP-1 analogs, its truncated forms or pharmaceutically acceptable salts and derivatives thereof, wherein one or more aminoacids are substituted with a non-coded aminoacid selected from the group comprising sugar aminoacids, furan aminoacids and dialkylated aminoacids.

The present invention further relates to a method of treating diabetes comprising administering a peptide selected from a group comprising GLP-1 analogs, its truncated forms or pharmaceutically acceptable salts and derivatives thereof, wherein one or more aminoacids are substituted with a non-coded aminoacid.

The present invention further relates to a method of treating diabetes comprising administering a peptide selected from a group comprising of GLP-1 analogs, its truncated forms or pharmaceutically acceptable salts and derivatives thereof, wherein one or more aminoacids are substituted with a non-coded aminoacid selected from the group comprising sugar aminoacids, furan aminoacids and dialkylated aminoacids.

The present invention further relates to the method of treating diabetes comprising administering a peptide selected from a group comprising of GLP-1 analogs, its truncated forms, or pharmaceutically acceptable salts and derivatives thereof, wherein one or more aminoacids are substituted with a non-coded aminoacid, optionally in combination with one or more anti-diabetic agents.

The present invention further relates to a peptide having the general formula:

His-Xaa¹-Xaa²-Gly-Thr-Phe-Thr-Ser-Xaa³-Xaa⁴-Xaa⁵⁻
Ser-Tyr-Xaa⁶-Glu-Gly-Gln-Ala-Ala-Lys-Xaa⁷-Phe-
Ile-Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention further relates to a peptide having the general formula:

X′aa¹-X′aa²-X′aa³-X′aa⁴-X′aa⁵-X′aa⁶-His-Xaa¹-Xaa²-
Gly-Thr-PheThr-Ser-Xaa³-Xaa⁴-Xaa⁵⁻Ser-Tyr-Xaa⁶-
Glu-Gly-Gln-Ala-Ala-Lys-Xaa⁷-PheIle-Xaa⁸-Trp-Leu-
Val-Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention further relates to a method of treating diabetes comprising administering a peptide having the general formula:

His-Xaa¹-Xaa²-Gly-Thr-Phe-Thr-Ser-Xaa³-Xaa⁴-Xaa⁵⁻
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention further relates to a method of treating diabetes comprising administering a peptide having the general formula:

X′aa¹-X′aa²-X′aa³-X′aa⁴-X′aa⁵-X′aa⁶-His-Xaa¹-Xaa²-
Gly-Thr-PheThr-Ser-Xaa³-Xaa⁴-Xaa⁵⁻Ser-Tyr-Xaa⁶-Glu-
Gly-Gln-Ala-Ala-Lys-Xaa⁷-Phe-Ile-Xaa⁸-Trp-Leu-Val-
Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylatcd aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention also relates to a pharmaceutical composition comprising a peptide having the general formula:

His-Xaa¹-Xaa²-Gly-Thr-Phe-Thr-Ser-Xaa³-Xaa⁴-Xaa⁵⁻
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted;

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylatcd aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention also relates to a pharmaceutical composition comprising a peptide having the general formula:

X′aa¹-X′aa²-X′aa³-X′aa⁴-X′aa⁵-X′aa6-His-Xaa¹-Xaa²-
Gly-Thr-PheThr-Ser-Xaa³-Xaa⁴-Xaa⁵⁻Ser-Tyr-Xaa⁶-Glu-
Gly-Gln-Ala-Ala-Lys-Xaa⁷-Phe-Ile-Xaa⁸-Trp-Leu-Val-
Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof and a pharmaceutically acceptable carrier.

The present invention also relates to a method of eliciting an agonist effect from a GLP-1 receptor comprising administering a peptide having the general formula:

His-Xaa¹-Xaa²-Gly-Thr-Phe-Thr-Ser-Xaa³-Xaa⁴-Xaa⁵⁻
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted;

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention also relates to a method of eliciting an agonist effect from a GLP-1 receptor comprising administering a peptide having the general formula:

X′aa¹-X′aa²-X′aa³-X′aa⁴-X′aa⁵-X′aa⁶-His-Xaa¹-Xaa²-
Gly-Thr-PheThr-Ser-Xaa³-Xaa⁴-Xaa⁵⁻Ser-Tyr-Xaa⁶-Glu-
Gly-Gln-Ala-Ala-Lys-Xaa⁷-Phe-Ile-Xaa⁸-Trp-Leu-Val-
Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention further relates to a peptide having the general formula:

His-Xaa¹-Xaa²-Gly-Thr-Phe-Thr-Ser-Xaa³-Xaa⁴-Xaa⁵⁻
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted;

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof, with the proviso that, if Xaa¹ is Furan aminoacid then Xaa² is Glu or deleted; Xaa³ is Asp or dialkylated aminoacid; Xaa⁴ is Val or dialkylated aminoacid, Xaa⁵ is Ser or Leu; Xaa⁶ is Leu or Gln; Xaa⁷ is Glu or Leu; Xaa⁸ is Ala or Glu or dialkylated aminoacid and Xaa⁹ is Gly or dialkylated aminoacid.

The present invention further relates to a peptide having the general formula:

X′aa¹-X′aa²-X′aa³-X′aa⁴-X′aa⁵-X′aa⁶-His-Xaa¹-
Xaa²-Gly-Thr-PheThr-Ser-Xaa³-Xaa⁴-Xaa⁵-Ser-Tyr-
Xaa⁶-Glu-Gly-Gln-Ala-Ala-Lys-Xaa⁷-Phe-Ile-Xaa⁸-
Trp-Leu-Val-Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof with the proviso that, if Xaa¹ is Furan aminoacid then X′aa¹ to X′aa⁶ are deleted; Xaa² is Glu or deleted; Xaa³ is Asp or dialkylated aminoacid; Xaa⁴ is Val or dialkylated aminoacid, Xaa⁵ is Ser or Leu; Xaa⁶ is Leu or Gln; Xaa⁷ is Glu or Leu; Xaa⁸ is Ala or Glu or dialkylated aminoacid and Xaa⁹ is Gly or dialkylated aminoacid.

The present invention further relates to a peptide having the general formula:

His-Xaa¹-Xaa²-Gly-Thr-Phe-Thr-Ser-Xaa³-Xaa⁴-Xaa⁵-
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, Sugar aminoacid, Furan aminoacid or Dialkylated aminoacid;

Xaa² is Glu, Sugar aminoacid, Furan aminoacid or Dialkylated aminoacid or deleted;

Xaa³ is Asp or Dialkylated aminoacid or deleted;

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable, salts and derivatives thereof, with the proviso that, if Xaa¹ is Sugar aminoacid then Xaa² is Glu or deleted.

The present invention further relates to a peptide having the general formula:

X′aa¹-X′aa²-X′aa³-X′aa⁴-X′aa⁵-X′aa⁶-His-Xaa¹-Xaa²-
Gly-Thr-PheThr-Ser-Xaa³-Xaa⁴-Xaa⁵-Ser-Tyr-Xaa⁶-
Glu-Gly-Gln-Ala-Ala-Lys-Xaa⁷-Phe-Ile-Xaa⁸-Trp-Leu-
Val-Lys-Xaa⁹-Xaa¹⁰

Wherein,

X′aa¹ is His or deleted;

X′aa² is Asp or deleted;

X′aa³ is Glu or deleted;

X′aa⁴ is Phe or deleted;

X′aa⁵ is Glu or deleted;

X′aa⁶ is Arg or deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof, with the proviso that, if Xaa¹ is Sugar aminoacid then X′aa¹ to X′aa⁶ are deleted; Xaa² is Glu or deleted.

The present invention further relates to a peptide having the general formula:

X′aa¹-X′aa²-X′aa³-X′aa⁴-X′aa⁵-X′aa⁶-His-Xaa¹-Xaa²-
Gly-Thr-Phe-Thr-Ser-Xaa³-Xaa⁴-Xaa⁵-Ser-Tyr-Xaa⁶-
Glu-Gly-Gln-Ala-Ala-Lys-Xaa⁷-Phe-Ile-Xaa⁸-Trp-Leu-
Val-Lys-Xaa⁹-Xaa¹⁰

Wherein,

X′aa1 to X′aa6 are deleted;

Xaa¹ is Gly, Sugar aminoacid, Furan aminoacid, Dialkylated aminoacid;

Xaa² is Glu, Sugar aminoacid, Furan aminoacid, Dialkylated aminoacid or deleted;

Xaa³ is Asp or Dialkylated aminoacid or deleted

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention further relates to a peptide having the general formula:

X′aa¹-X′aa²-X′aa³-X′aa⁴-X′aa⁵-X′aa⁶-His-Xaa¹-Xaa²-
Gly-Thr-Phe-Thr-Ser-Xaa³-Xaa⁴-Xaa⁵-Ser-Tyr-Xaa⁶-
Glu-Gly-Gln-Ala-Ala-Lys-Xaa⁷-Phe-Ile-Xaa⁸-Trp-Leu-
Val-Lys-Xaa⁹-Xaa¹⁰

Wherein, X′aa¹ to X′aa⁶ are deleted;

Xaa¹ is Gly, 2-amino-1-O-methyl-2-deoxy-α-D-glucopyranuronic acid, 5-aminomethyl-furan-2 carboxylic acid, α-aminoisobutyric acid;

Xaa² is Glu, 2-amino-1-O-methyl-2-deoxy-α-D-glucopyranuronic acid, 5-aminomethyl-furan-2 carboxylic acid, α-aminoisobutyric acid or deleted;

Xaa³ is Asp or α-aminoisobutyric acid or deleted

Xaa⁴ is Val or α-aminoisobutyric acid or deleted;

Xaa⁵ is Ser or Leu;

Xaa⁶ is Leu or Gln;

Xaa⁷ Glu or Leu;

Xaa⁸ is Ala or Glu or α-aminoisobutyric acid or deleted;

Xaa⁹ is Gly or α-aminoisobutyric acid or deleted;

Xaa¹⁰ is one or more natural aminoacid or one or more amidated natural aminoacid or deleted;

or pharmaceutically acceptable salts and derivatives thereof.

The present invention further relates to a peptide having the general formula:

His-Xaa¹-Xaa²-Gly-Thr-Phe-Thr-Ser-Xaa³-Xaa⁴-Xaa⁵-
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰

Wherein,

Xaa¹ is Gly, 2-amino-1-O-methyl-2-deoxy-α-D-glucopyranuronic acid, 5-aminomethyl-furan-2 carboxylic acid, α-aminoisobutyric acid;

Xaa² is Glu, 2-amino-1-O-methyl-2-deoxy-α-D-glucopyranuronic acid, 5-aminomethyl-furan-2 carboxylic acid, α-aminoisobutyric acid or deleted;

Xaa³ is Asp or Dialkylated aminoacid or deleted;

Xaa⁴ is Val or Dialkylated aminoacid or deleted;

Xaa⁵ is Ser or Leu;

Xaa⁶ is Leu or Gln;

Xaa⁷ is Glu or Leu;

Xaa⁸ is Ala or Glu or Dialkylated aminoacid or deleted;

Xaa⁹ is Gly or Dialkylated aminoacid or deleted;

Xaa¹⁰ is one or more natural aminoacid or one or more amidated natural aminoacid or deleted;

or pharmaceutically acceptable salts and derivatives thereof.

The present invention further relates to a peptide having the formula:

(Seq ID No. 01)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly-NH₂

or pharmaceutically acceptable salts and derivatives thereof, wherein Xaa¹ is Furan aminoacid.

The present invention further relates to a peptide having the formula:

(Seq ID No. 01)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly-NH₂

or pharmaceutically acceptable salts and derivatives thereof, wherein Xaa¹ is 5-aminomethyl-furan-2 carboxylic acid.

The present invention further relates to a peptide having the formula:

(Seq ID No. 02)
His-Xaa¹-Gly-Thr-Phe-Thr-Ser-Asp-Val-Ser-Ser-Tyr-
Leu-Glu-Gly-Gln-Ala-Ala-Lys-Glu-Phe-Ile-Ala-Trp-
Leu-Val-Lys-Gly-Arg-Gly-NH₂

or pharmaceutically acceptable salts and derivatives thereof, wherein Xaa¹ is Furan aminoacid.

The present invention further relates to a peptide having the formula:

(Seq ID No. 02)
His-Xaa¹-Gly-Thr-Phe-Thr-Ser-Asp-Val-Ser-Ser-Tyr-
Leu-Glu-Gly-Gln-Ala-Ala-Lys-Glu-Phe-Ile-Ala-Trp-
Leu-Val-Lys-Gly-Arg-Gly-NH₂

or pharmaceutically acceptable salts and derivatives thereof, wherein Xaa¹ is 5-aminomethyl-furan-2 carboxylic acid.

The present invention further relates to a peptide having the formula:

(Seq ID No. 03)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-NH₂

or pharmaceutically acceptable salts and derivatives thereof, wherein Xaa¹ is Furan aminoacid.

The present invention further relates to a peptide having the formula:

(Seq ID No. 03)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-NH₂

or pharmaceutically acceptable salts and derivatives thereof, wherein Xaa¹ is 5-aminomethyl-furan-2 carboxylic acid.

The present invention further relates to a peptide having the formula:

(Seq ID No. 04)
His-Xaa¹-Glu-Gly-Thr-Phe-Thr-Ser-Asp-Xaa²-Ser-Ser-
Tyr-Leu-Glu-Gly-Gln-Ala-Ala-Lys-Glu-Phe-Ile-Xaa³-
Trp-Leu-Val-Lys-Gly-Arg-NH₂

or pharmaceutically acceptable salts and derivatives thereof, wherein Xaa¹ is furan amino acid and; Xaa² and Xaa³ are Dialkylated aminoacid.

The present invention further relates to a peptide having the formula:

(Seq ID No. 04)
His-Xaa¹-Glu-Gly-Thr-Phe-Thr-Ser-Asp-Xaa²-Ser-Ser-
Tyr-Leu-Glu-Gly-Gln-Ala-Ala-Lys-Glu-Phe-Ile-Xaa³-
Trp-Leu-Val-Lys-Gly-Arg-NH₂

or pharmaceutically acceptable salts and derivatives thereof, wherein Xaa¹ is 5-aminomethyl-furan-2 carboxylic acid and; Xaa² and Xaa³ are α-aminoisobutyric acid.

The present invention further relates to a peptide having the formula:

(Seq ID No. 05)
His-Gly-Glu-Gly-Thr-Phe-Thr-Ser-Asp-Xaa¹-Ser-Ser-
Tyr-Leu-Glu-Gly-Gln-Ala-Ala-Lys-Glu-Phe-Ile-Xaa²-
Trp-Leu-Val-Lys-Gly-Arg-NH₂

or pharmaceutically acceptable salts and derivatives thereof, wherein Xaa¹ and Xaa² are dialkylated aminoacid.

The present invention further relates to a peptide having the formula:

(Seq ID No. 05)
His-Gly-Glu-Gly-Thr-Phe-Thr-Ser-Asp-Xaa¹-Ser-
Ser-Tyr-Leu-Glu-Gly-Gln-Ala-Ala-Lys-Glu-Phe-
Ile-Xaa²-Trp-Leu-Val-Lys-Gly-Arg-NH₂

or pharmaceutically acceptable salts and derivatives thereof, wherein Xaa¹ and Xaa² are α-aminoisobutyric acid.

The present invention further relates to a peptide having the formula:

(Seq ID No. 06)
His-Xaa¹-Glu-Gly-Thr-Phe-Thr-Ser-Asp-Val-Ser-
Ser-Tyr-Leu-Glu-Gly-Gln-Ala-Ala-Lys-Glu-Phe-
Ile-Ala-Trp-Leu-Val-Lys-Gly-Arg-NH₂

or pharmaceutically acceptable salts and derivatives thereof, wherein Xaa¹ is sugar aminoacid.

The present invention further relates to a peptide having the formula:

(Seq 1D No. 06)
His-Xaa¹-Glu-Gly-Thr-Phe-Thr-Ser-Asp-Val-Ser-
Ser-Tyr-Leu-Glu-Gly-Gln-Ala-Ala-Lys-Glu-Phe-
Ile-Ala-Trp-Leu-Val-Lys-Gly-Arg-NH₂

or pharmaceutically acceptable salts and derivatives thereof, wherein Xaa¹ is 2-amino-1-O-methyl-2-deoxy-α-D-glucopyranuronic acid.

The present invention further relates to a peptide having the formula:

(Seq ID No. 07)
His-Xaa¹-Gly-Thr-Phe-Thr-Ser-Asp-Val-Ser-Ser-
Tyr-Leu-Glu-Gly-Gln-Ala-Ala-Lys-Glu-Phe-Ile-
Ala-Trp-Leu-Val-Lys-Gly-Arg-NH₂

or pharmaceutically acceptable salts and derivatives thereof, wherein Xaa¹ is sugar aminoacid.

The present invention further relates to a peptide having the formula:

(Seq ID No. 07)
His-Xaa¹-Gly-Thr-Phe-Thr-Ser-Asp-Val-Ser-Ser-
Tyr-Leu-Glu-Gly-Gln-Ala-Ala-Lys-Glu-Phe-Ile-
Ala-Trp-Leu-Val-Lys-Gly-Arg-NH₂

or pharmaceutically acceptable salts and derivatives thereof, wherein Xaa¹ is 2-amino-1-O-methyl-2-deoxy-α-D-glucopyranuronic acid.

The present invention further relates to a peptide having the formula:

(Seq ID No. 08)
His-Xaa¹-Gly-Thr-Phe-Thr-Ser-Asp-Val-Ser-Ser-
Tyr-Leu-Glu-Gly-Gln-Ala-Ala-Lys-Glu-Phe-Ile-
Ala-Trp-Leu-Val-Lys-Gly-Arg-NH₂

or pharmaceutically acceptable salts and derivatives thereof, wherein Xaa¹ is furan aminoacid.

The present invention further relates to a peptide having the formula:

(Seq ID No. 08)
His-Xaa¹-Gly-Thr-Phe-Thr-Ser-Asp-Val-Ser-Ser-
Tyr-Leu-Glu-Gly-Gln-Ala-Ala-Lys-Glu-Phe-Ile-
Ala-Trp-Leu-Val-Lys-Gly-Arg-NH₂

or pharmaceutically acceptable salts and derivatives thereof, wherein Xaa¹ is 5-aminomethyl-furan-2 carboxylic acid.

The present invention further relates to a peptide having the formula:

(Seq ID No. 09)
His-Xaa¹-Glu-Gly-Thr-Phe-Thr-Ser-Asp-Xaa²-Ser-
Ser-Tyr-Leu-Glu-Gly-Gln-Ala-Ala-Lys-Glu-Phe-Ile-
Xaa³-Trp-Leu-Val-Lys-Gly-Arg-Gly-NH₂

or pharmaceutically acceptable salts and derivatives thereof, wherein Xaa¹ is furan amino acid and; Xaa² and Xaa³ are dialkylated aminoacid.

The present invention further relates to a peptide having the formula:

(Seq ID No. 09)
His-Xaa¹-Glu-Gly-Thr-Phe-Thr-Ser-Asp-Xaa²-Ser-
Ser-Tyr-Leu-Glu-Gly-Gln-Ala-Ala-Lys-Glu-Phe-Ile-
Xaa³-Trp-Leu-Val-Lys-Gly-Arg-Gly-NH₂

or pharmaceutically acceptable salts and derivatives thereof, wherein Xaa¹ is 5-aminomethyl-furan-2 carboxylic acid and; Xaa² and Xaa³ are α-aminoisobutyric acid.

The present invention further relates to a peptide having the formula:

(Seq ID No. 10)
His-Xaa¹-Gly-Thr-Phe-Thr-Ser-Asp-Xaa²-Ser-Ser-
Tyr-Leu-Glu-Gly-Gln-Ala-Ala-Lys-Glu-Phe-Ile-
Xaa³-Trp-Leu-Val-Lys-Gly-Arg-Gly-NH₂

or pharmaceutically acceptable salts and derivatives thereof, wherein Xaa¹ is furan aminoacid and; Xaa² and Xaa³ are dialkylated aminoacid.

The present invention further relates to a peptide having the formula:

(Seq ID No. 10)
His-Xaa¹-Gly-Thr-Phe-Thr-Ser-Asp-Xaa²-Ser-Ser-
Tyr-Leu-Glu-Gly-Gln-Ala-Ala-Lys-Glu-Phe-Ile-
Xaa³-Trp-Leu-Val-Lys-Gly-Arg-Gly-NH₂

or pharmaceutically acceptable salts and derivatives thereof, wherein Xaa¹ is 5-aminomethyl-furan-2 carboxylic acid and; Xaa² and Xaa³ are α-aminoisobutyric acid.

The present invention further relates to a peptide having the formula:

(Seq ID No. 11)
His-Xaa¹-Gly-Thr-Phe-Thr-Ser-Asp-Val-Ser-Ser-
Tyr-Leu-Glu-Gly-Gln-Ala-Ala-Lys-Glu-Phe-Ile-
Ala-Trp-Leu-Val-Lys-Xaa²-Arg-NH₂

or pharmaceutically acceptable salts and derivatives thereof, wherein Xaa¹ is furan aminoacid and; Xaa² is dialkylated aminoacid.

The present invention further relates to a peptide having the formula:

(Seq ID No. 11)
His-Xaa¹-Gly-Thr-Phe-Thr-Ser-Asp-Val-Ser-Ser-
Tyr-Leu-Glu-Gly-Gln-Ala-Ala-Lys-Glu-Phe-Ile-
Ala-Trp-Leu-Val-Lys-Xaa²-Arg-NH₂

or pharmaceutically acceptable salts and derivatives thereof, wherein Xaa¹ is 5-aminomethyl-furan-2 carboxylic acid and; Xaa² is α-aminoisobutyric acid.

The present invention further relates to a peptide having the formula:

(Sequence ID No. 12)
His-Xaa¹-Gly-Thr-Phe-Thr-Ser-Asp-Val-Leu-Ser-
Tyr-Gln-Glu-Gly-Gln-Ala-Ala-Lys-Leu-Phe-Ile-
Glu-Trp-Leu-Val-Lys-Gly-Arg-NH₂

or pharmaceutically acceptable salts and derivatives thereof, wherein Xaa¹ is furan aminoacid.

The present invention further relates to a peptide having the formula:

(Sequence ID No. 12)
His-Xaa¹-Gly-Thr-Phe-Thr-Ser-Asp-Val-Leu-Ser-
Tyr-Gln-Glu-Gly-Gln-Ala-Ala-Lys-Leu-Phe-Ile-
Glu-Trp-Leu-Val-Lys-Gly-Arg-NH₂

or pharmaceutically acceptable salts and derivatives thereof, wherein Xaa¹ is 5-aminomethyl-furan-2 carboxylic acid.

The peptides of the present invention may be prepared using any technique known in the art. The peptides of the present invention may be prepared by chemical synthesis as well as recombinant synthesis; preferably the peptides can be synthesized using chemical synthesis i.e. solid phase techniques. More preferably, the peptides of the present invention were synthesized by solid phase synthesis (Fmoc Solid Phase peptide synthesis—A Practical approach” Edited by W. C Chan and P. D White, Oxford University Press, USA, 2004).

More preferably, the peptides of the present invention can be synthesized by solid phase techniques using Fmoc Strategy on automatic peptide synthesizer (Applied Biosystems 433A Peptide Synthesizer) at 0.25 mmol scale. The peptides may be assembled from C-terminus to N-terminus. Peptides amidated at C-terminus may be synthesized using Rink Amide Resin.

The chemical moieties can be used to protect reactive side chains of the peptides during synthesis procedure. The N-terminal amino group can be protected by 9-fluorenylmethoxycarbonyl (Fmoc) group. The functional side chain of aminoacids may be protected as described in standard text (“Peptide synthesis protocols”—Edited by M. W. Pennington and B. M. Bunn, Humana Press, 1995)

The peptide on resin may be cleaved using standard cleavage mixture after assembling. The crude peptide may be lyophilized and further purified and analyzed by HPLC.

The term “peptide” according to the present invention means natural or synthetic compounds containing two or more amino acids. The “peptide” according to the present invention may specifically mean analogs or truncated form of GLP-1.

The peptides according to the present invention includes, but not limited to, peptides as such, pharmaceutically acceptable salts of peptides, peptide analogs, homologous peptides, fusion peptides, derivatized peptide, such as, for example, a peptide modified to contain one or more-chemical moieties other than an amino acid.

GLP-1 as used in the invention would mean GLP-1 (1-37), GLP-1(7-34), GLP-1(7-35), GLP-1 (6-37), GLP-1(7-37), GLP-1(7-37) amide, GLP-1(7-36), GLP-1(7-36) amide, their truncated forms, analogs or a pharmaceutically acceptable salt and derivatives thereof, wherein one or more aminoacids are substituted with a non-coded aminoacids selected from the group comprising sugar aminoacids, furan aminoacids and dialkylated aminoacids.

The term “analog or analogue” as used herein means, but not limited to, a modified peptide wherein one or more aminoacid residues of the peptide have been substituted by other aminoacid residues and/or wherein one or more aminoacid residues have been deleted from the peptide and/or wherein one or more aminoacid residues have been added to the peptide.

The term “aminoacid/aminoacid residues” used above may be genetically encoded aminoacids (Natural aminoacid), non-genetically encoded (also referred to as Non-coded aminoacid) aminoacids, synthetic L-aminoacids or D-enantiomer/s of all of the above or pharmaceutically acceptable salts/derivatives thereof.

“Non-coded aminoacid” according to the present invention means, but not limited to, any non-natural aminoacids which are not encoded by a genetic code. Preferably, the non-coded aminoacid can be selected from, but not limited to, the group comprising sugar aminoacid, furan aminoacid, dialkylated aminoacid and combinations thereof.

“Sugar aminoacid or (Saa)” according to the present invention are basically, but not limited to, the hybrids of carbohydrate and aminoacids where amino and carboxyl functional groups have been incorporated at the two termini of regular 2,5- or 2,6-anhydro sugar frameworks. Preferably the sugar aminoacid is 2-amino-1-O-methyl-2-deoxy-α-D-glucopyranuronic acid.

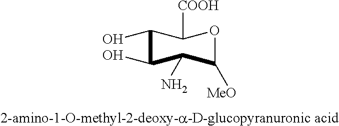

“Furan aminoacid or (Faa)” according to the present invention means, but not limited to, the hybrid aminoacids where amino and carboxyl functional groups have been incorporated at the two side chains of Furan ring. Preferably the furan aminoacid is 5-aminomethyl-furan-2 carboxylic acid.

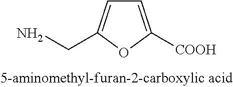

“Dialkylated aminoacids or (Daa)” according to the present invention means, but not limited to, aminoacids which are alkylated at C-α position. Preferably the dialkylated aminoacid is C-α,α-dimethyl glycine or α-Aminoisobutyric acid (Aib); α,α-diethyl glycine; α,α-di-n-propyl glycine; α,α-n-butyl glycine and the corresponding cyclic amino acids such as 1-aminocyclopentane 1-carboxylic acid, 1-aminocyclohexane 1-carboxylic acid, 1-aminocycloheptane 1-carboxylic acid and 1-aminocyclooctane 1-carboxylic acid.

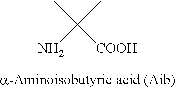

It must be noted that as used in the specification and the appended claims, the singular forms ‘a’, ‘an’ and ‘the’ include plural references unless the context clearly indicates otherwise.

The peptides of the present invention acts as GLP-1 analog and in view of the various activities associated with GLP-1, the peptides that are described herein can be used generally to achieve one or more of the following biological activities: 1) stimulate insulin release, 2) reduce blood glucose levels, 3) increase plasma insulin levels, 4) stimulate transcription of β-cell-specific genes (e.g., GLUT-1 transporter, insulin receptor and hexokinase-1), 5) increase β-cell mass by inhibiting β-cell apoptosis and increasing β-cell proliferation and replication, 6) induce satiety thereby reducing food intake and promoting weight loss, 7) reduce gastric secretion, 8) delay gastric emptying, and 9) reduce gastric motility.

The peptides of the present invention can be used to treat diabetes and other related disorders. The peptides of the present invention can be used for eliciting an agonist effect from a GLP-1 receptor.

Formulation and Route of Administration

The compounds of the present invention may be administered to a subject per se or in the form of a pharmaceutical composition. Pharmaceutical compositions comprising the peptides of the present invention may be manufactured by any means such as conventional mixing, dissolving, granulating, dragee-making, levigating, emulsifying, encapsulating, entrapping, lyophilizing processes or the like. Pharmaceutical compositions may be formulated in conventional manner using one or more physiologically acceptable carriers, diluents, excipients or auxiliaries which facilitate processing of the active peptides or peptide analogues into preparations which can be used pharmaceutically. Proper formulation is dependent upon the route of administration chosen.

Suitable pharmaceutical carriers are described in the most recent edition of Remington's Pharmaceutical Sciences, A. Osol, a standard reference text in this field.

The pharmaceutical compositions of the present invention may be administered by any means that enables the active agent to reach the site of action in the body of a mammal. The peptides of the present invention can be administered by any route of administration known in the art. The various routes of administration includes, but not limited to, topical, parenteral, transmucosal, oral, buccal, rectal, inhalation, nasal, vaginal or sublingual.

“Pharmaceutically acceptable salts and derivatives” according to the present invention are those salts and derivatives which substantially retain the biological activity of the free bases. The pharmaceutically acceptable salt and derivatives includes salts and derivatives which can be prepared according to the person skilled in the art.

Derivatives of the GLP-1 analog peptides can further mean, but not limited to, that the peptide may he amidated, acylated, acetylated, sulfated, phosphorylated, glycosylated, oxidized, esterified, and polyethylene glycol-modified and which substantially retain the biological activity of the free bases.

The peptides of the present invention will generally be used in an amount effective to achieve the intended purpose. For use to treat or prevent GLP-1 related disorders, the peptides of the present invention or pharmaceutical compositions thereof are administered or applied in a therapeutically effective amount. By therapeutically effective amount, is meant an amount effective to ameliorate or prevent the symptoms, or prolong the survival of, the patient being treated. Determination of a therapeutically effective amount is well within the capabilities of those skilled in the art, especially in light of the detailed disclosure provided herein.

The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the peptides which are sufficient to maintain therapeutic effect. Usual patient dosages for administration by injection range from about 0.001 mg/kg/day to 5 g/kg/day, preferably in the range of about 0.01 mg/kg/day to about 5 mg/kg/day. Dosage amount and interval may be adjusted individually to achieve plasma levels which are effective in ameliorating the pathological condition.

In cases of local administration or selective uptake, the effective local concentration of the peptides may not be related to plasma concentration. One, having skill in the art, will be able to optimize therapeutically effective local dosages without undue experimentation.

The amount of peptide administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. Throughout this application, various publications and patents are referenced with patents by number and other publications by author and year. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### General Methodology and Results

Synthesis of Peptides

The novel peptides of the present invention were synthesized by solid phase synthesis techniques using Fmoc Strategy on automatic peptide synthesizer (Applied Biosystems 433A Peptide Synthesizer) at 0.25 mmol scale. The peptides were assembled from C-terminus to N-terminus. Peptides amidated at C-terminus were synthesized using Rink Amide Resin. The resin employed for synthesis was Rink Amide MBHA resin LL (100-200 mesh) procured from Novabiochem (Substitution 0.34 mmol/g resin).

The chemical moieties were used to protect reactive side chains of the peptides during synthesis procedure. The N-terminal amino group was protected by 9-fluorenylmethoxycarbonyl (Fmoc) group. The Alanine, Glycine, Isoleucine, Leucine, phenylalanine, Valine were used unprotected. The side chains of lysine and Tryptophan were Boc protected. Aspartate and glutamate residues were used with t-butyl ester (OtBu) protection. The side chain of Glutamine and Histidine were trityl (trt) protected. Serine, Threonine and Tyrosine were used with t-Butyl (tBu) protection. Arginine residue was used with 2,2,4,6,7-pentamethyldihydrobenzofuran-5-sulfonyl (Pbf) protection.

The activating reagents used for coupling amino acids to the resin include HBTU (O-Benzotriazole-N,N,N′,N′-tetramethyl-uronium-hexofluoro-phosphate)/HOBt and DIEA (Diisopropyl ethylamine). The coupling reaction was carried out in NMP (N-Methyl-2-pyrrolidine). After the assembly of the peptide chain was completed the peptide-resin was washed with methanol and dried. The peptide was cleaved from resin by treatment with a cleavage mixture consisting of trifluoroacetic acid, crystalline phenol, thioanisol, ethanedithiol and de-ionized water for 2-3 hrs at room temperature. The crude peptide was obtained by precipitation with cold dry ether, filtered, dissolved and lyophilized.

The resulting crude peptide was purified by preparative HPLC using a Phenomenex C18 (250×22.1) reverse phase column using a gradient of 0.1% TFA in Acetonitrile and water. The eluted fractions were reanalyzed on Analytical HPLC system (Shimadzu Corporation, Japan) using a Phenomenex C18 (250×4.6) reverse phase column. Acetonitrile was evaporated and the fractions were lyophilized to obtain the pure peptide. The identity of each peptide was confirmed by mass spectra.

### EXAMPLE 1

### Synthesis of Seq ID No: 01

The peptide was synthesized using solid phase synthesis (as described above). The sequence was started on 0.125 mmol scale; 0.8 g of peptide resin was obtained. After cleavage and lyophilization, 340 mg of crude peptide was obtained. It was further purified by HPLC and characterized by mass spectra. The calculated mass was ˜3405 and the observed mass was (M/3) was 1135.25.

### EXAMPLE 2

### Synthesis of Seq ID No: 02

The peptide was synthesized using solid phase synthesis (as described above). The sequence was started on 0.125 mmol scale; 0.8 g of peptide resin was obtained. After cleavage and lyophilization, 342 mg of crude peptide was obtained. It was further purified by HPLC and characterized by mass spectra. The calculated mass was ˜3279 and the observed mass (M/3) was 1092.2

### EXAMPLE 3

### Synthesis of Seq ID No: 03

The peptide was synthesized using solid phase synthesis (as described above). The sequence was started on 0.25 mmol scale; 1.677 g of peptide resin was obtained. After cleavage and lyophilization, 496 mg of crude peptide was obtained. It was further purified by HPLC and characterized by mass spectra. The calculated mass was ˜3349 and the observed mass was (M/3) was 1116.23

### EXAMPLE 4

### Synthesis of Seq ID No: 04

The peptide was synthesized using solid phase synthesis (as described above). The sequence was started on 0.25 mmol scale, 1.723 g of peptide resin was obtained. After cleavage and lyophilization, 608 mg of crude peptide was obtained. It was further purified by HPLC and characterized by mass spectra. The calculated mass was ˜3668 and the observed mass was (M/3) was 1222.73

### EXAMPLE 5

### Synthesis of Seq ID No: 05

The peptide was synthesized using solid phase synthesis (as described above). The sequence was started on 0.25 mmol scale; 1.8 g of peptide resin was obtained. After cleavage and lyophilization, 724 mg of crude peptide was obtained. It was further purified by HPLC and characterized by mass spectra. The calculated mass was ˜3284 and the observed mass was (M/3) was 1094.87

### EXAMPLE 6

### Synthesis of Seq ID No: 06

The peptide was synthesized using solid phase synthesis (as described above). The sequence was started on 0.125 mmol scale; 0.81 g of peptide resin was obtained. After cleavage and lyophilization, 312.2 mg of crude peptide was obtained. It was further purified by HPLC and characterized by mass spectra. The calculated mass was ˜3415 and the observed mass was (M/3) was 1138.43

### EXAMPLE 7

### Synthesis of Seq ID No: 07

The peptide was synthesized using solid phase synthesis (as described above). The sequence was started on 0.125 mmol scale; 0.89 g of peptide resin was obtained. After cleavage and lyophilization, 295 mg of crude peptide was obtained. It was further purified by HPLC and characterized by mass spectra. The calculated mass was ˜3286 and the observed mass was (M/3) was 1097.3

### EXAMPLE 8

### Synthesis of Seq ID No: 08

The peptide was synthesized using solid phase synthesis (as described above). The sequence was started on 0.125 mmol scale; 0.84 g of peptide resin was obtained. After cleavage and lyophilization, 328 mg of crude peptide was obtained. It was further purified by HPLC and characterized by mass spectra. The calculated mass was ˜3220 and the observed mass was (M/3) was 1074.4

### EXAMPLE 9

### Synthesis of Seq ID No: 09

The peptide was synthesized using solid phase synthesis (as described above). The sequence was started on 0.125 mmol scale; 0.81 g of peptide resin was obtained. After cleavage and lyophilization, 312.2 mg of crude peptide was obtained. It was further purified by HPLC and characterized by mass spectra. The calculated mass was ˜3405 and the observed mass was (M/3) was 1136.9.

### EXAMPLE 10

### Synthesis of Seq ID No: 10

The peptide was synthesized using solid phase synthesis (as described above). The sequence was started on 0.125 mmol scale; 0.81 g of peptide resin was obtained. After cleavage and lyophilization, 312.2 mg of crude peptide was obtained. It was further purified by HPLC and characterized by mass spectra. The calculated mass was ˜3276 and the observed mass was (M/3) was 1093.7.

### EXAMPLE—11

### Synthesis of Seq ID No: 11

The peptide was synthesized using solid phase synthesis (as described above). The sequence was started on 0.17 mmol scale; 1.10 g of peptide resin was obtained. After cleavage and lyophilization ˜512 mg of crude peptide was obtained. It was further purified by HPLC and characterized by mass spectra. The calculated mass was ˜3377 and the observed mass (M/3) was 1127.3.

### EXAMPLE—12

### Synthesis of Seq ID No: 12

The peptide was synthesized using solid phase synthesis (as described above). The sequence was started on 0.25 mmol scale; 1.71 g of peptide resin was obtained. After cleavage and lyophilization ˜694 mg of crude peptide was obtained. It was further purified by HPLC and characterized by mass spectra. The calculated mass was ˜3370 and the observed mass was (M/3) was 1124.0.

### EXAMPLE 13

### Biological Activity of Peptides

In Vitro Receptor Functional Assay of Peptides

cAMP is a ubiquitous cellular second messenger that is critical component of a signal transduction pathway linking membrane receptors and their ligands to the activation of internal cellular enzymatic activity and gene expression. cAMP is synthesized from ATP by membrane bound adenylate cyclase in a very regulated manner. In its simpler form, binding of a ligand, such as hormone or drug, to its specific G protein coupled receptor can either stimulate or inhibit adenylate cyclase depending on the GPCR being activated. In case of GLP-1 R which is abound to Gs type protein the GPCR gets stimulated and cAMP gets synthesized as an effect to external stimuli. Therefore the relative concentration of cAMP serves as a means to monitor the activity of GPCRs at the cell surface.

### REFERENCES

1: Hormone action. Chapter 31, in Biochemistry. Zubay, G, editor, 2^{nd} ed., Macmillan Publishing company, New York, 1045-1085 (1988)

2: Rotella, D. P. Phosphodiesterase 5 inhibitors: current status and potential application. Nature Reviews Drug Discover 1, 674-682 (2002)

3: Doyle, M. E., Greig, N. H., Holloway, H. W., Betkey, J. A.

In Vitro Receptor Functional Assay:

CHO/GLP-1R Cell Culture—CHO/GLP-1R Cells were maintained in Ham's F-12 medium containing 10% Fetal bovine serum and 1% penicillin-streptomycin antibiotic solution at 37° C. in a 5% CO₂ incubator.

Determination of biological activity of Peptides: 5×10⁴ CHO/GLP-1R Cells per well were seeded in 96 wells plate and grown overnight. Cells were washed twice with PBS (pH-7.2) and incubated with 100 μl Ham's F-12 serum free medium supplemented with 0.1% BSA for 2 hr at 37° C. in 5% CO₂ incubator. Cells were then incubated in 100 μl Ham's F-12 serum free medium supplemented with 0.1% BSA with 1 mM IBMX in the presence as well as absence of the peptides as per present invention in different concentration (10 nM, 1 nM, 100 pM & 10 pM). The reaction was stopped 30 min later by washing the intact cells three times with ice cold PBS (pH-7.2). The intracellular cAMP was extracted by incubating the cells in 100 μl 0.1N HCl at room temperature for 20 minutes. Mixture was dissociated by pipetting up and down until the suspension became homogenous, transferred to centrifuge tube and centrifuged at 800 rpm for 10 minutes. The supernatant was assayed using Kookaburra Cyclic AMP Kit Catalog No. 133-16475 (SAPPHIRE BIOSCENCE). GLP-1 activity potency (EC₅₀) to receptor on CHO/GLP-1 cells calculated by using Graph pad prizm software.

Reagent Preparation

1. Tris Buffer: Diluted the contents of one vial of Tris Buffer with 90 ml of MQ (Milli Q) water.

2. Cyclic AMP Alkaline Phosphates Tracer: Reconstituted the cAMP AP tracer with 6 ml of tris buffer. Vortex to mix.

3. Cyclic AMP Antiserum: Reconstituted the cAMP Antiserum with 6 ml of tris Buffer. Vortex to mix.

4. DEA Buffer: Diluted the 2.5 ml vial of DEA buffer concentrate to a final volume of 25 ml with MQ (Milli Q) water.

5. Wash Buffer: Diluted the 5 ml vial of wash buffer to a final volume of 750 ml with MQ (Milli Q) water.

100 μl sample/standard (GLP-1 parent i.e. GLP-1(7-36) parent and GLP-1(7-37) parent) were pipetted out into the appropriate wells according to template, 50 μl cAMP alkaline phosphate tracer and cAMP antiserum was added then plate was incubated for two hours at room temperature on shaker. Dissolved 5 pNPP tablets in 25 ml DEA buffer. Wells were emptied and washed five times with wash buffer and added 200 μl pNPP solution to each well, plate was covered and allowed to develop in dark for 90 minutes. Plate was read at a wavelength 405 nm and EC₅₀ value was calculated using Graph Pad Prism Software.

TABLE 1
Receptor Functional assay

| Peptide | EC50 nm |
|---|---|
| GLP-1(7-36) parent | 0.290 |
| GLP-1(7-37) parent | 0.0503 |
| Seq ID No. 01 | 0.103 |
| Seq ID No. 02 | 0.043 |
| Seq ID Mo. 03 | 0.061 |
| Seq ID No. 04 | 0.072 |
| Seq ID No. 05 | 0.083 |
| Seq ID No. 06 | 0.838 |
| Seq ID No. 07 | 0.056 |
| Seq ID No. 08 | 0.601 |
| Seq ID No. 09 | 1.00 |
| Seq ID No. 10 | 3.38 |
| Seq ID No. 11 | 0.102 |
| Seq ID No. 12 | 2.24 |

### EXAMPLE 14

### DPP-IV Resistant Assay

20 μM of peptides of the present invention (Sequence IDs—01, 02, 03, 04, 05, 06, 11, 12 and 13) were incubated with 60 mU/2 ml of DPP-IV (Sigma-Aldrich) enzyme at room temperature (22-24° C.) in 20 mM Tris HCl buffer, pH 8.0. The aliquots were analyzed for the amount of undegraded peptide concentration at different time intervals by RP-HPLC. The peak area integration was used to determine the amount of undegraded peptide (U.S. Pat. No. 7,067,488). T_{1/2} of each peptide was determined from the graph plotted between peak area of undegraded peptide vs. time. The peak area of DPP-IV untreated peptide in above mentioned buffer condition of same concentration was considered as time zero. The T_{1/2} thus determined is shown in Table 2. The peptides of the present invention i.e. (Sequence IDs—01, 02, 03, 04, 05, 06, 11, 12 and 13) were found to have better half lives than the parent GLP-1 molecules i.e. GLP-1(7-37) parent and GLP-1(7-36) parent.

TABLE 2
DPP-IV Enzyme Resistant Assay

| | T_{1/2} in Hrs & (Percentage |
|---|---|
| Peptide | degradation in hrs) |
| GLP-1(7-37)parent | 2.84 Hrs |
| GLP-1(7-36)parent | 2.66 Hrs |
| Seq ID No. 01 | >72 Hrs (25% in 24 hrs) |
| Seq ID No. 02 | >24 Hrs(43.62% in 24 hrs) |
| Seq ID No. 03 | >72 Hrs(13% in 72 hrs) |
| Seq ID No. 04 | >24 Hrs (12.87% in 24 hrs) |
| Seq ID No. 05 | 24.66 Hrs |
| Seq ID No. 06 | 4.33 Hrs |
| Seq ID No. 11 | >24 Hrs(20% in 24 hrs) |
| Seq ID No. 12 | >24 Hrs(24.93% in 24 hrs) |

### EXAMPLE 15

### NEP 24.11 Resistant Assay

20 μM GLP-1 analogs were incubated with 1 mg/ml of NEP 24.11 (Calbiochem) enzyme at room temperature (37° C.) in 50 mM Hepes Buffer, 50 mM NaCl, pH 7.4. The aliquots were analyzed for the amount of undegraded peptide concentration at different time intervals using RP-HPLC by peak area integration. T_{1/2} of each peptide was determined from the graph plotted between peak area of undegraded peptide vs time. The peak area of NEP 24.11 untreated peptide in above mentioned buffer condition of same concentration was considered as time zero. The T_{1/2} thus determined is shown in Table 3.

The half life of Seq ID No. 05 and Seq ID No. 07 was found to be better than the parent GLP-1 peptide i.e. GLP-1(7-36) parent and the half life of the peptides of Seq ID No.01, Seq ID No.03 and Seq ID No.12 was found to be comparable with the GLP-1(7-36) parent peptide.

TABLE 3
NEP 24.11 Degradation Assay

| | | % Degradation |
|---|---|---|
| Peptide | T_{1/2} (Hrs) | (after 54 hrs) |
| GLP-1(7-36)parent | 27 | 98.28 |
| Seq ID No. 1 | 29 | 92.57 |
| Seq ID No. 3 | 29 | 96.75 |
| Seq ID No. 4 | >56 | 25.98 |
| Seq ID No. 5 | 49 | 59.4 |
| Seq ID No. 12 | 27.5 | 93.52 |

### EXAMPLE—16

### In-Vivo Screening of Peptides in C57BL/6 Mice (Oral Glucose Tolerance Test or OGTT)

General Methodology:

A volume of 100 μl of peptides of present invention (2 μg per mouse) were injected intraperitoneally into 6 hr fasted C57BL/6 normal male/female mice (4-6 wk). After 6 h of fasting, fasting glucose level was be assessed by using a Glucometer (Accu-Chek) from tail vein or the retro-orbital sinus. Glucose was given orally at the ratio 1 mg/g body wt. per mouse 5 min after the administration of peptides. Blood glucose levels were assessed at different time points i.e. at 0, 15, 30, 60, 120 and 180 min.

The graph was plotted for blood glucose (mg/dl) level against time. Control negative graph shows the blood glucose level when only placebo (saline) injected.

### EXAMPLE: 16.1

OGTT Experiment in Male Mice:

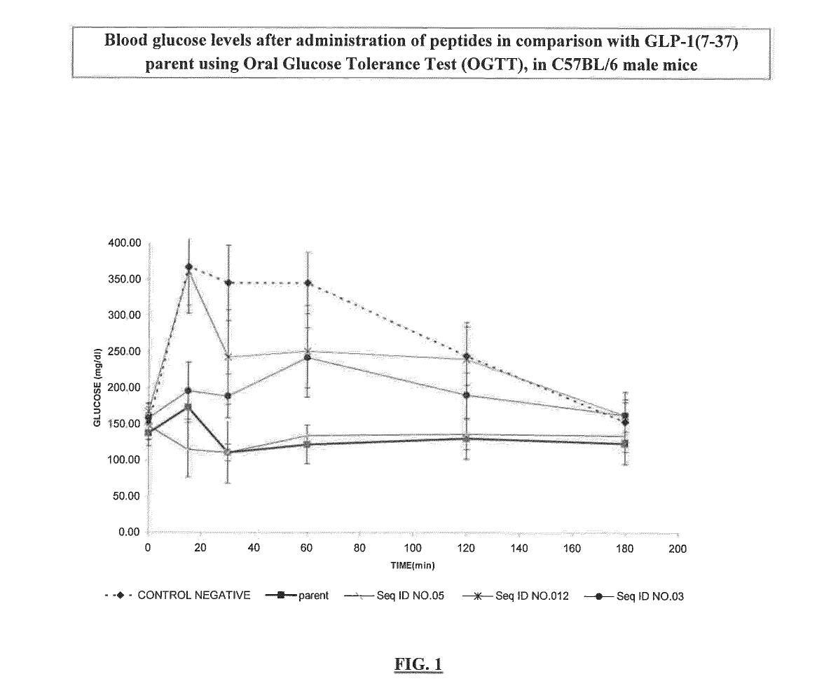

Oral Glucose Tolerance Test as described in above was conducted using GLP-1(7-37) parent (referred in FIG. 1 as parent) and peptides of Seq ID No.03, Seq ID No.05, and Seq ID No.12 in C57BL/6 male mice. The results are shown in FIG. 1 that describes blood glucose level (mg/dl) of male mice after administration of GLP-1(7-37) parent and peptides at different time points. The saline solution was administered to the male mice that served as control negative.

### EXAMPLE: 16.2

OGTT Experiment in Female Mice:

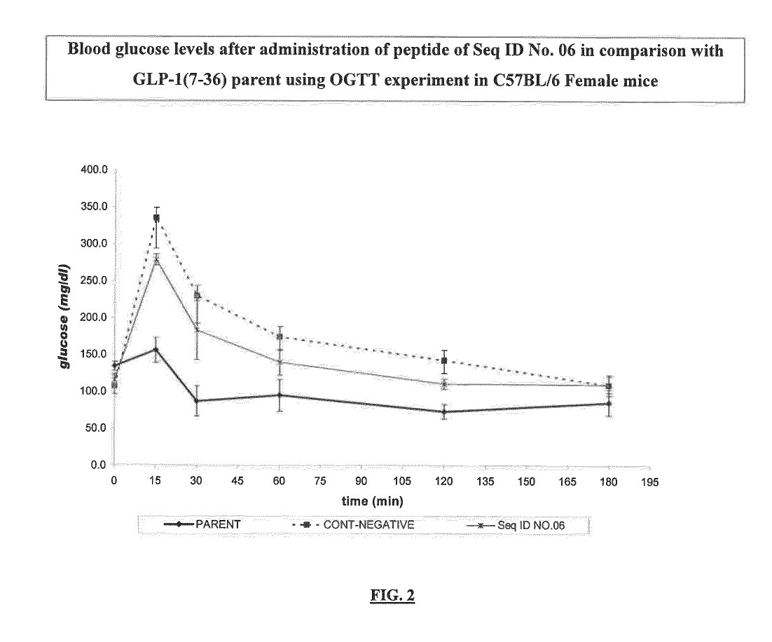

Oral Glucose Tolerance Test was conducted with Peptide of Seq ID No.06 and GLP-1(7-36) parent molecule in female mice with the same procedure as described in Example 16. The results are shown in FIG. 2, which illustrates blood glucose level (mg/dl) of female mice after administration of GLP-1(7-36) parent and peptide of Seq ID No.06 at different time points. The saline solution was administered to the female mice as control negative.

### EXAMPLE: 16.3

OGTT Experiment in Male Mice

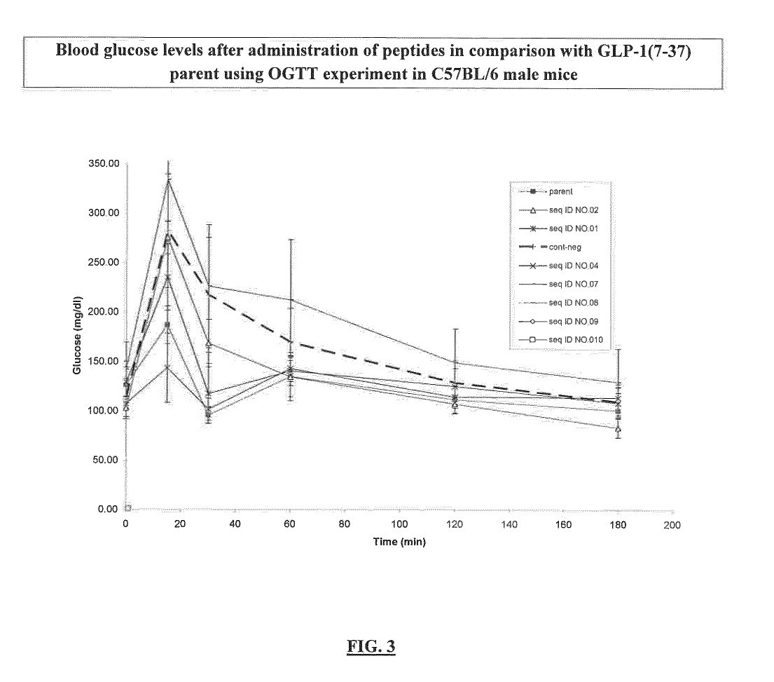

OGTT was performed with peptides of Seq ID No.01, Seq ID No.02, Seq ID No.04, Seq ID No.07, Seq ID No.08, Seq ID No.09 and Seq ID No.010 along with GLP-1(7-37) parent at same dose in male mice (as per method described above in Example—16). The results are shown in FIG. 3 which describes blood glucose level (mg/dl) of male mice after administration of GLP-1(7-37) parent and peptides of Seq ID No.01, Seq ID No.02, Seq ID No.04, Seq ID No.07, Seq ID No.08, Seq ID No.09 and Seq ID No.10 at different time points. The saline solution was administered to the male mice as control negative.

Results:

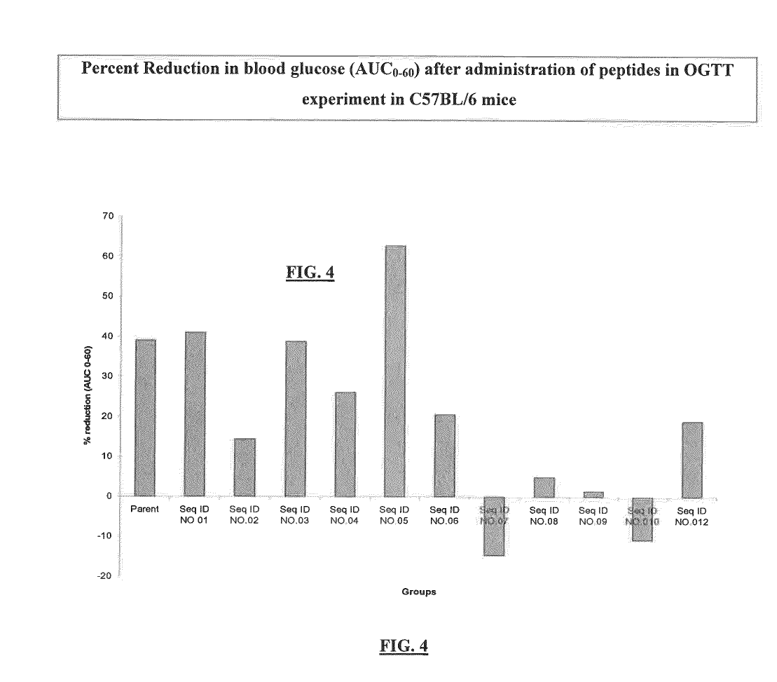

The area under the blood glucose concentration curve was obtained over a 60-minute period and percent reduction in AUC was calculated over the AUC of negative control. The percent reduction in blood glucose (AUC₀₋₆₀) of different peptides (i.e. Sequence IDs No. 01 to 10 & 12) [according to OGTT Experiment in C57BL/6 mice] is shown in FIG. 4. The results clearly showed that peptides of Seq ID No.05 showed better activity than GLP-1(7-37) parent peptide and Seq ID No 01 and Seq ID No.03 showed comparable activity to the GLP-1(7-37) parent peptide.

### EXAMPLE—17

### OGTT and Dose Response Effect of Peptides

Peptides of Seq ID No.02, Seq ID No.03, Seq ID No.04, and Seq ID No.11 (in different doses 25 nmol, 100 nmol, 200 nmol and 400 nmol/kg) were injected intraperitoneally to 6 hrs fasted C57/BL6 normal male mice (6 weeks old) 5 minutes prior to an oral glucose administration. Glucose (1 g/kg) was given by oral route, and blood sample were taken by tail vein. Blood glucose levels were measured at different time intervals such as t=0, 15, 30, 60 and 120 minutes using one-touch glucometer. The saline solution was administered to the male mice as control negative.

Results:

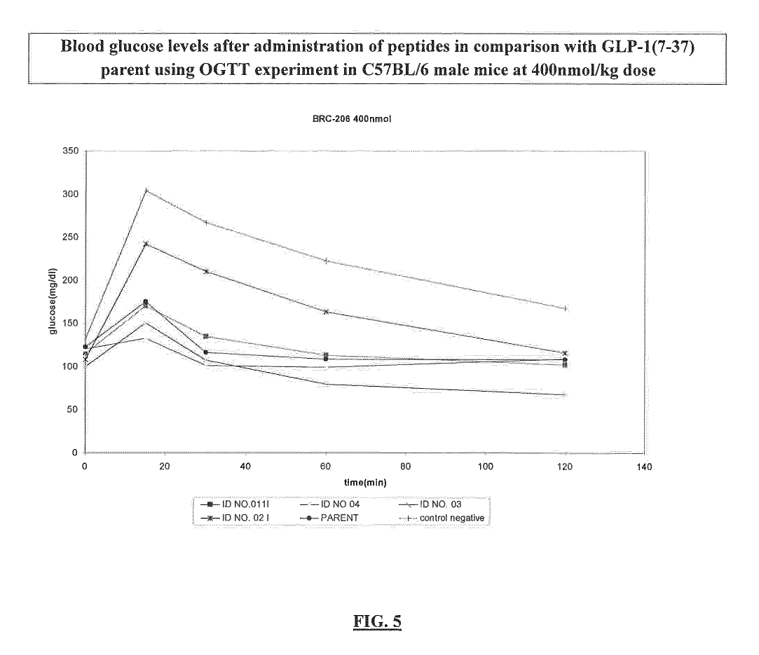

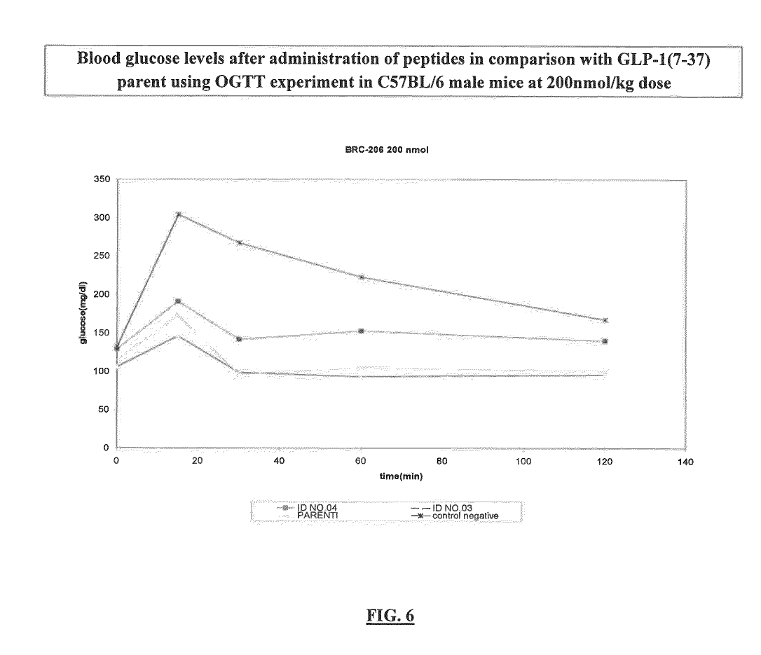

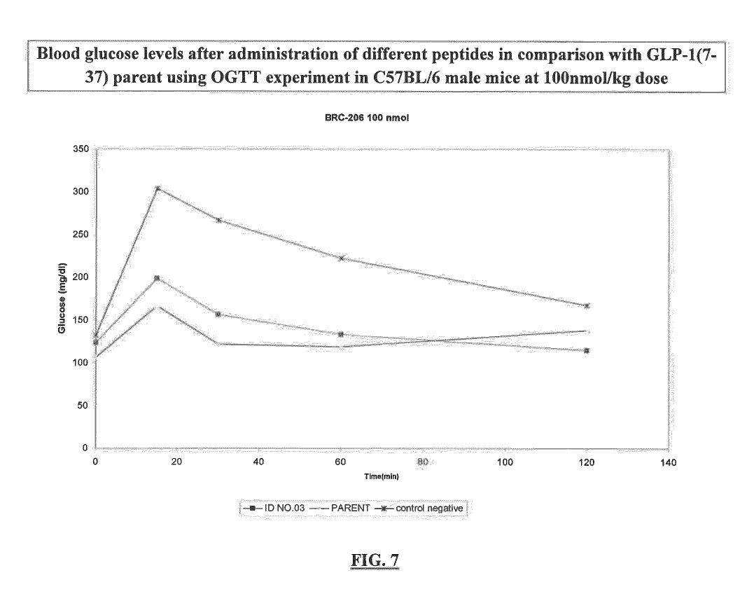

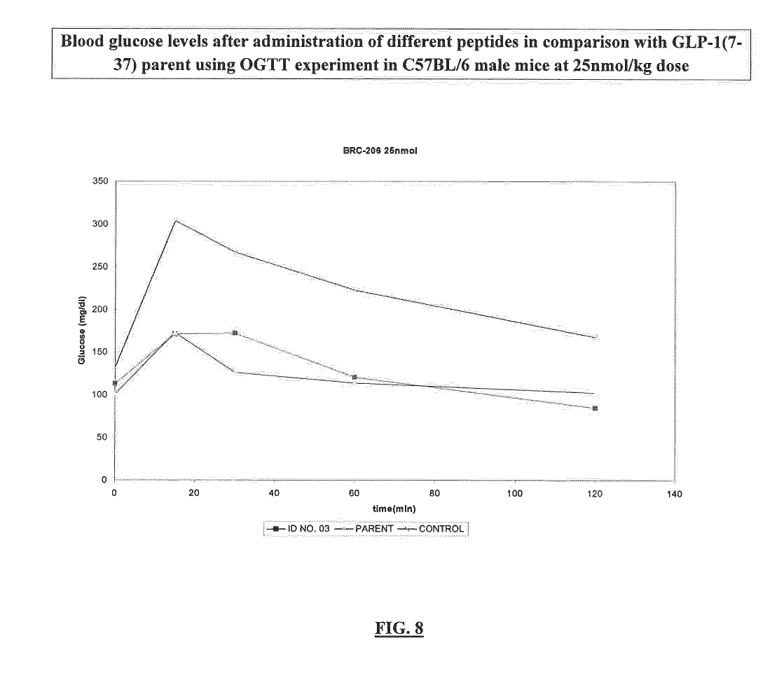

The results are shown in FIG. 5, FIG. 6, FIG. 7 and FIG. 8. The description of the Figures is as follows:

FIG. 5: provides blood glucose level (mg/dl) of mice after administration of GLP-1(7-37) parent and peptides of present invention (Seq ID No.02, Seq ID No.03, Seq ID No.04, and Seq ID No.011) at 400 nmol/kg, at different time points i.e. t=0, 15, 30, 60 and 120 minutes.

FIG. 6 provides blood glucose level (mg/dl) of mice after administration of GLP-1(7-37) parent and peptides of present invention (Seq ID No.03 and Seq ID No.04) at 200 nmol/kg, at different time points i.e. t=0, 15, 30, 60 and 120 minutes.

FIG. 7 provides blood glucose level (mg/dl) of mice after administration of GLP-1(7-37) parent peptide and peptide of present invention (Seq ID No.03) at 100 nmol/kg, at different time points i.e. t=0, 15, 30, 60 and 120 minutes.

FIG. 8, provides blood glucose level (mg/dl) of mice after administration of GLP-1(7-37) parent peptide and peptide of present invention (Seq ID No.03) at 25 nmol/kg, at different time points i.e. t=0, 15, 30, 60 and 120 minutes.

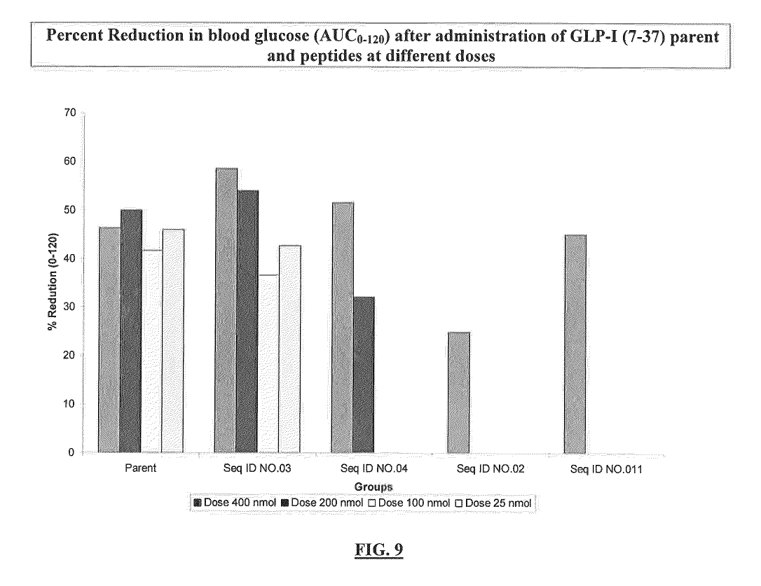

The area under the blood glucose concentration curve was obtained over a 120-minute period and percent reduction was calculated over AUC of control negative group). The percent reduction in blood glucose (AUC₀₋₁₂₀) of different peptides (as per present invention i.e. Sequence IDs No. 02 to 04 & 11) [according to OGTT Experiment in C57BL/6 mice] is shown in FIG. 9. The figure clearly shows that the peptide of Seq. ID No. 03 showed better activity to GLP-1(7-37) parent at the same dose.

### EXAMPLE—18

### OGTT and Dose Response Effect of Peptide of Seq ID No. 01

C57/BL6 male mice were fasted for 6 hrs. After 6 h of fasting, blood was drawn from a small tail clip for glucose level determination. The peptide of Seq ID No. 01 was injected intraperitoneally at different doses i.e. 100 nmol, 25 nmol, 10 nmol, 5 nmol and 1 nmol/kg to the fasted male mice. Glucose was given orally at the ratio 1 mg/g body wt. per mouse 5 min after the administration of peptide. Blood glucose levels were determined at different time intervals i.e. at 0, 15, 30, 60, and 120 min during a period of 2 hours using a portable glucometer. The saline solution was used as control negative in the experiment.

Results:

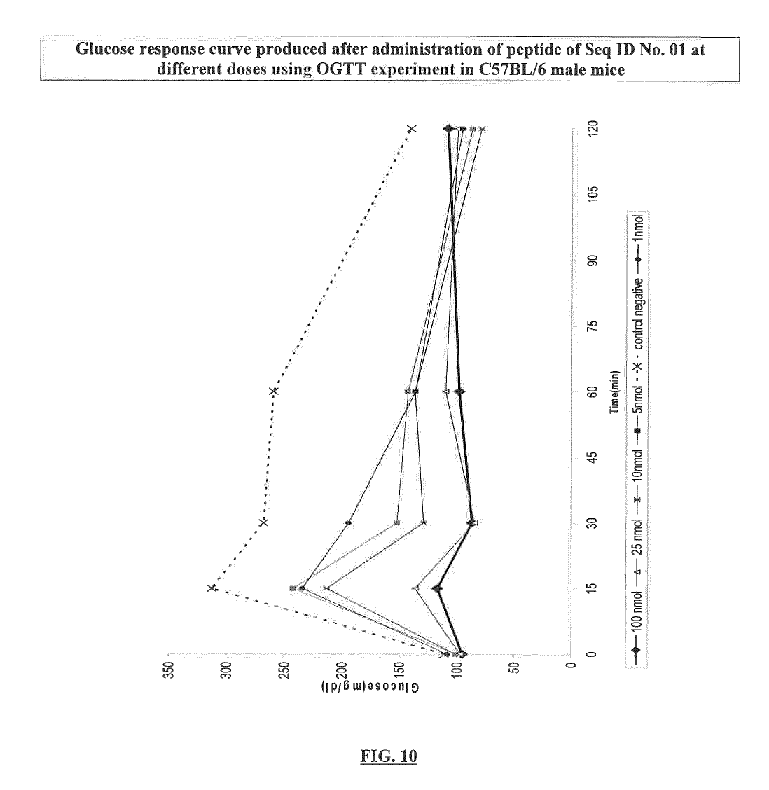

The results are shown in FIG. 10 which provides blood glucose level (mg/dl) of male mice after administration of Seq ID No. 01 at different doses (100 nmol, 25 nmol, 10 nmol, 5 nmol and 1 nmol/kg) at different time points i.e. at 0, 15, 30, 60, and 120 min.

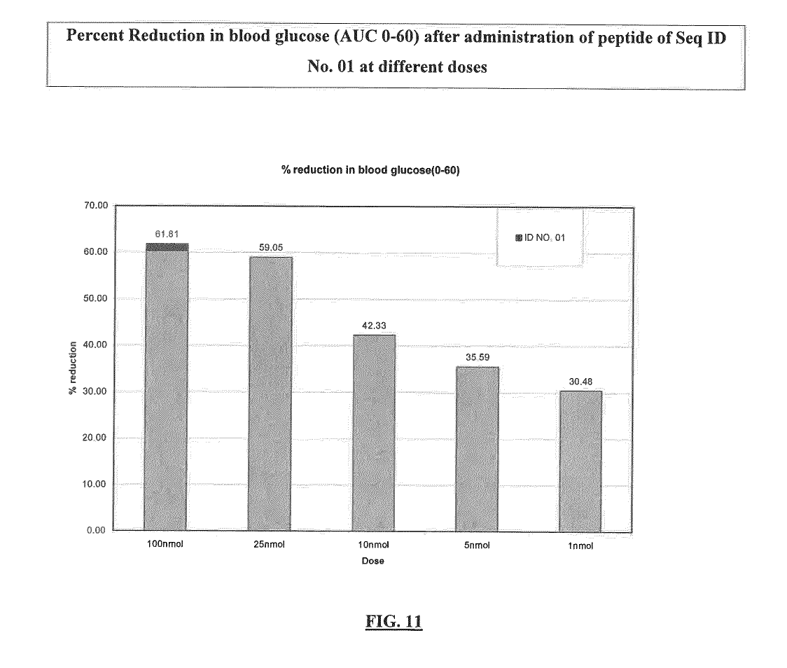

The area under the blood glucose concentration curve was obtained over a 60-minute period and percent reduction in AUC was calculated over the AUC of control negative group. The percent reduction in blood glucose (AUC₀₋₆₀) of the peptide (i.e. Sequence IDs No. 01) at different doses (100 nmol, 25 nmol, 10 nmol, 5 nmol and 1 nmol/kg) [according to OGTT Experiment in C57BL/6 male mice] is shown in FIG. 11.

The results from the dose response experiment indicated that there was no significant difference between 100 nmol and 25 nmol doses. So the optimum dose was found to be 25 nmol/kg for Seq ID No. 01.

### EXAMPLE—19

### Anti-Diabetic Activity of Peptide in db/db Mice

Animals of two different age groups (6-8 and 10-12 weeks old db/db mice) were included in this study. 6-8 week old mice were chosen as pre-diabetic mice and 10-12 week old as the full-diabetic mice. The preventive effect of peptide of Seq. ID No. 01 on the progression of diabetes in pre-diabetic mice was examined. The effect of peptide of Seq. ID No. 01 in full-diabetic mice was also studied.

Long term efficacy study of peptide of Seq ID No. 01 (25 nmol/kg) was performed for 42 days in 6-8 weeks old mice and 28 days for 10-12 weeks old mice at the same dose. Blood glucose levels were measured on day 0, 14, 28 and 42. Test peptide of Seq ID No. 01 was injected i.p. according to body weight and the feed was removed. Mice were not fed till 4 hrs following injection of the peptide on the day of blood sample testing. Normal chow was given after the 4 hr time point. Blood glucose level was measured at time t=1 hr, and t=4 hr on day 0, 14, 28 and 42. Test compound/vehicle (saline solution i.e. control negative) to be administered at 0 hr. The animals were subjected to daily i.p. doses of peptide of Seq ID No. 01 for 42 days.

Results:

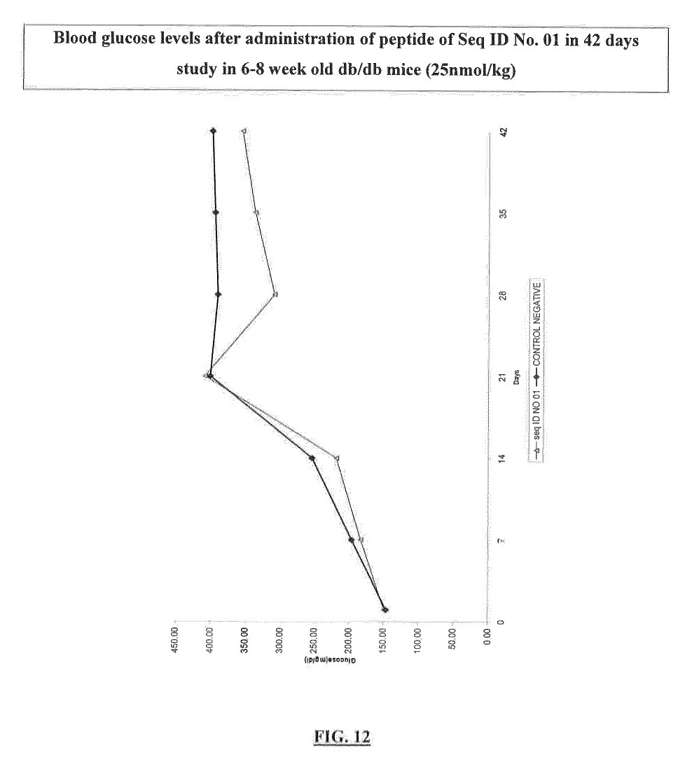

The results are shown in FIG. 12 which describes blood glucose level (mg/dl) of mice after administration of Saline solution (control negative) and peptide of Seq ID No. 01 in 6-8 weeks old db/db mice at different time points. The results from the 42 days study indicated that peptide of Seq ID No. 01 delayed the progression of diabetes as compared to control negative group (saline) in 6-8 weeks old db/db mice.

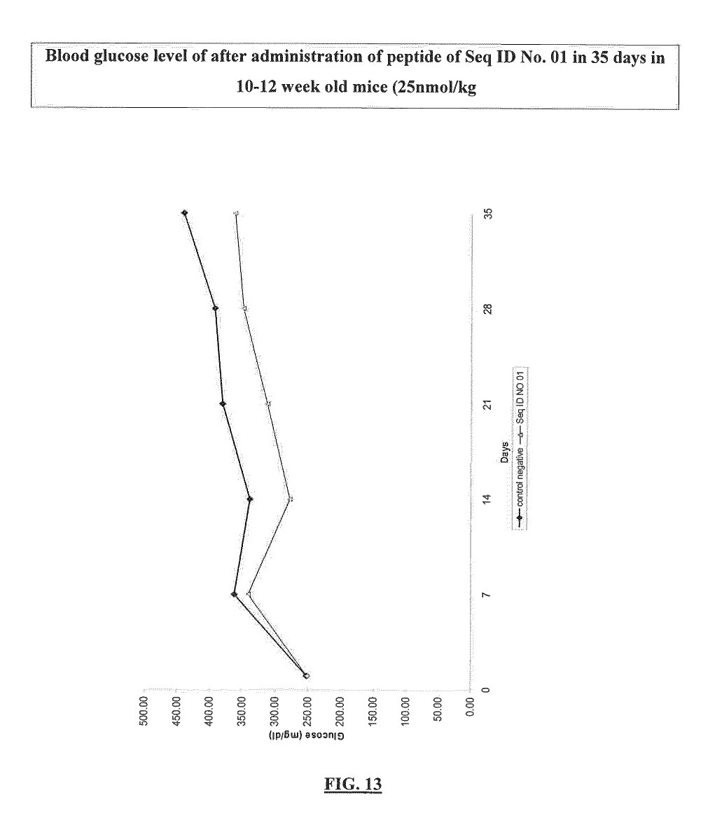

The results are shown in FIG. 13 that describes blood glucose level (mg/dl) of GLP-1(7-37) parent and peptide of Seq ID No. 01 in 10-12 weeks old db/db mice at different time points. The results from 28 days study indicated that peptide of Seq. ID No. 01 showed better activity as compared to control negative group (saline solution) 10-12 weeks old db/db mice.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

1. A peptide having the general formula:
(Seq ID No. 13)
His-Xaa¹-Xaa²-Gly-Thr-Phe-Thr-Ser-Xaa³-Xaa⁴-Xaa⁵-
Ser-Tyr-Xaa⁶-Glu-Gly-Gln-Ala-Ala-Lys-Xaa⁷-Phe-Ile-
Xaa⁸-Trp-Leu-Val-Lys-Xaa⁹-Xaa¹⁰
Wherein,
Xaa¹ is Sugar aminoacid, Furan aminoacid or Dialkylated aminoacid;
Xaa² is Glu, Sugar aminoacid, Furan aminoacid or Dialkylated aminoacid or deleted;
Xaa³ is Asp or Dialkylated aminoacid or deleted;
Xaa⁴ is Val or Dialkylated aminoacid or deleted;
Xaa⁵ is Ser or Leu;
Xaa⁶ is Leu or Gln;
Xaa⁷ is Glu or Leu;
Xaa⁸ is Ala or Glu or Dialkylated aminoacid or deleted;
Xaa⁹ is Gly or Dialkylated aminoacid or deleted;
Xaa¹⁰ is one or more natural aminoacid or one or more amidated natural aminoacid or deleted; or pharmaceutically acceptable salts and derivatives thereof, with the proviso that when Xaa¹ is a Dialkylated amino acid then Xaa¹⁰ is not an Amidated aminoacid.

2. A peptide according to claims 1 wherein Xaa¹ is Furan aminoacid.

3. A peptide according to claims 1 wherein Xaa¹ is Sugar aminoacid.

4. (canceled)

5. A peptide according to claim 1 wherein if Xaa¹ is Furan amino acid then Xaa² is Glu or deleted; Xaa³ is Asp or dialkylated aminoacid; Xaa⁴ is Val or dialkylated aminoacid, Xaa⁵ is Ser or Leu; Xaa⁶ is Leu or Gln; Xaa⁷ is Glu or Leu; Xaa⁸ is Ala or Glu or dialkylated aminoacid and Xaa⁹ is Gly or dialkylated aminoacid.

6. A peptide according to claim 1 wherein if Xaa¹ is Sugar aminoacid then Xaa² is Glu or deleted.

7. A peptide selected from the group consisting of:
(Seq ID No. 1)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly-NH₂;
(Seq ID No. 2)
His-Xaa¹-Gly-Thr-Phe-Thr-Ser-Asp-Val-Ser⁻-Ser-Tyr-
Leu-Glu-Gly-Gln-Ala-Ala-Lys-Glu-Phe-Ile-Ala-Trp-
Leu-Val-Lys-Gly-Arg-Gly-NH₂;
(Seq ID No. 3)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-NH₂;
(Seq ID No. 4)
His-Xaa¹-Glu-Gly-Thr-Phe-Thr-Ser-Asp-Xaa²-Ser-
Ser-Tyr-Leu-Glu-Gly-Gln-Ala-Ala-Lys-Glu-Phe-Ile-
Xaa³-Trp-Leu-Val-Lys-Gly-Arg-NH₂;
(Seq ID No. 8)
His-Xaa¹-Gly-Thr-Phe-Thr-Ser-Asp-Val-Ser⁻-Ser-Tyr-
Leu-Glu-Gly-Gln-Ala-Ala-Lys-Glu-Phe-Ile-Ala-Trp-
Leu-Val-Lys-Gly-Arg-NH₂;
(Seq ID No. 9)
His-Xaa¹-Glu-Gly-Thr-Phe-Thr-Ser-Asp-Xaa²-Ser-
Ser-Tyr-Leu-Glu-Gly-Gln-Ala-Ala-Lys-Glu-Phe-Ile-
Xaa³-Trp-Leu-Val-Lys-Gly-Arg-Gly-NH₂;
(Seq ID No. 10)
His-Xaa¹-Gly-Thr-Phe-Thr-Ser-Asp-Xaa²-Ser-Ser-
Tyr-Leu-Glu-Gly-Gln-Ala-Ala-Lys-Glu-Phe-Ile-
Xaa³-Trp-Leu-Val-Lys-Gly-Arg-Gly-NH₂;
(Seq ID No. 11)
His-Xaa¹-Gly-Thr-Phe-Thr-Ser-Asp-Val-Ser-Ser-Tyr-
Leu-Glu-Gly-Gln-Ala-Ala-Lys-Glu-Phe-Ile-Ala-Trp-
Leu-Val-Lys-Xaa²-Arg-NH₂;
(Sequence ID No. 12)
His-Xaa¹-Gly-Thr-Phe-Thr-Ser-Asp-Val-Leu-Ser-Tyr-
Gln-Glu-Gly-Gln-Ala-Ala-Lys-Leu-Phe-Ile-Glu-Trp-
Leu-Val-Lys-Gly-Arg-NH₂;
or pharmaceutically acceptable salts and derivatives thereof, wherein Xaa¹ is furan aminoacid and; Xaa² and Xaa³ are dialkylated aminoacid.

8. A peptide selected from the group consisting of:
(Seq ID No. 6)
His-Xaa¹-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-NH₂;
(Seq ID No. 7)
His-Xaa¹-Gly-Thr-Phe-Thr-Ser-Asp-Val-Ser⁻-Ser-Tyr-
Leu-Glu-Gly-Gln-Ala-Ala-Lys-Glu-Phe-Ile-Ala-Trp-
Leu-Val-Lys-Gly-Arg-NH₂
or pharmaceutically acceptable salts and derivatives thereof, wherein Xaa¹ is Sugar aminoacid.

9. A peptide of the formula:
(Seq ID No. 5)
His-Gly-Glu-Gly-Thr-Phe-Thr-Ser-Asp-Xaa¹-Ser-Ser-
Tyr-Leu-Glu-Gly-Gln-Ala-Ala-Lys-Glu-Phe-Ile-Xaa²-
Trp-Leu-Val-Lys-Gly-Arg-NH₂
or pharmaceutically acceptable salts and derivatives thereof, wherein Xaa¹ and Xaa² are Dialkylated aminoacid.

10. A peptide according to claim 1, wherein Furan aminoacid is 5-aminomethyl-furan-2 carboxylic acid.

11. A peptide according to claim 1, wherein Sugar aminoacid is 2-amino-1-O-methyl-2-deoxy-α-D-glucopyranuronic acid.

12. A peptide according to claim 1, wherein Dialkylated aminoacid is α-Aminoisobutyric acid.

13. A pharmaceutical composition comprising a peptide according to claim 1, or pharmaceutically acceptable salts and derivatives thereof and a pharmaceutically acceptable carrier.

14. A method of treating diabetes comprising administering a peptide according to claim 1, or pharmaceutically acceptable salts and derivatives thereof.

15. A method of eliciting an agonist effect from a GLP-1 receptor in a subject in need thereof comprising administering to said subject a peptide according to claim 1, or pharmaceutically acceptable salts and derivatives thereof.

16. A method of treating diabetes comprising administering a peptide according to claim 1, optionally in combination with one or more anti-diabetic agents.

17. A peptide having the general formula:
X′aa¹-X′aa²-X′aa³-X′aa⁴-X′aa⁵-X′aa⁶-His-Xaa¹-
Xaa²-Gly-Thr-Phe-Thr-Ser-Xaa³-Xaa⁴-Xaa⁵⁻Ser-Tyr-
Xaa⁶-Glu-Gly-Gln-Ala-Ala-Lys-Xaa⁷-Phe-Ile-Xaa⁸-
Trp-Leu-Val-Lys-Xaa⁹-Xaa¹⁰
Wherein,
X′aa¹ is His or deleted;
X′aa² is Asp or deleted;
X′aa³ is Glu or deleted;
X′aa⁴ is Phe or deleted;
X′aa⁵ is Glu or deleted;
X′aa⁶ is Arg or deleted;
Xaa¹ is Sugar aminoacid, Furan aminoacid or Dialkylated aminoacid;
Xaa² is Glu, Sugar aminoacid, Furan aminoacid or Dialkylated aminoacid or deleted;
Xaa³ is Asp or Dialkylated aminoacid or deleted;
Xaa⁴ is Val or Dialkylated aminoacid or deleted;
Xaa⁵ is Ser or Leu;
Xaa⁶ is Leu or Gln;
Xaa⁷ is Glu or Leu;
Xaa⁸ is Ala or Glu or Dialkylated aminoacid or deleted;
Xaa⁹ is Gly or Dialkylated aminoacid or deleted;
Xaa¹⁰ is one or more natural aminoacid or one or more amidated natural aminoacid or deleted;
or pharmaceutically acceptable salts and derivatives thereof, with the proviso that when Xaa¹ is a dialkylated amino acid then Xaa¹⁰ is not an amidated aminoacid, with the proviso that when Xaa¹ is a dialkylated amino acid then Xaa¹⁰ is not an amidated aminoacid.


SEQUENCE LISTING (13 sequences)

SEQ ID NO: 1 - protein, 31 aa, unknown
  Synthetic peptide
HXEGTFTSDVSSYLEGQAAKEFIAWLVKGRG

SEQ ID NO: 2 - protein, 30 aa, Unknown
  Synthetic Peptide
HXGTFTSDVSSYLEGQAAKEFIAWLVKGRG

SEQ ID NO: 3 - protein, 30 aa, Unknown
  Synthetic Peptide
HXEGTFTSDVSSYLEGQAAKEFIAWLVKGR

SEQ ID NO: 4 - protein, 30 aa, Unknown
  Synthetic Peptide
HXEGTFTSDXSSYLEGQAAKEFIXWLVKGR

SEQ ID NO: 5 - protein, 30 aa, Unknown
  Synthetic Peptide
HGEGTFTSDXSSYLEGQAAKEFIXWLVKGR

SEQ ID NO: 6 - protein, 30 aa, Unknown; identical to SEQ ID NO: 3
  Synthetic Peptide

SEQ ID NO: 7 - protein, 29 aa, Unknown
  Synthetic Peptide
HXGTFTSDVSSYLEGQAAKEFIAWLVKGR

SEQ ID NO: 8 - protein, 29 aa, Unknown
  Synthetic Peptide
HXGTFTSDVSSYLEGQAAKEFIAWLVKGR

SEQ ID NO: 9 - protein, 31 aa, Unknown
  Synthetic Peptide
HXEGTFTSDXSSYLEGQAAKEFIXWLVKGRG

SEQ ID NO: 10 - protein, 30 aa, Unknown
  Synthetic Peptide
HXGTFTSDXSSYLEGQAAKEFIXWLVKGRG

SEQ ID NO: 11 - protein, 29 aa, Unknown
  Synthetic Peptide
HXGTFTSDVSSYLEGQAAKEFIAWLVKXR

SEQ ID NO: 12 - protein, 29 aa, Unknown
  Synthetic Peptide
HXGTFTSDVLSYQEGQAAKLFIEWLVKGR

SEQ ID NO: 13 - protein, 30 aa, Unknown
  Synthetic Peptide
HXXGTFTSXXXSYXEGQAAKXFIXWLVKXX